SEQUENCE LISTING (180 sequences)

SEQ ID NO: 1 - DNA, 59 bp, Artificial Sequence
  Description of Artificial Sequence primer YLR243w-S1
atgtctcgcgttggtgtcatggtattaggacctgcaggtgcagctgaagcttcgtacgc

SEQ ID NO: 2 - DNA, 59 bp, Artificial Sequence
  Description of Artificial Sequence primer YLR272c-S1
tttgggtctcgcactttctcagtcttccaactaatttctccagctgaagcttcgtacgc

SEQ ID NO: 3 - DNA, 59 bp, Artificial Sequence
  Description of Artificial Sequence primer YLR275w-S1
ccgttttatcatgtcgtatgtttgatcttaaccatttttacagctgaagcttcgtacgc

SEQ ID NO: 4 - DNA, 62 bp, Artificial Sequence
  Description of Artificial Sequence primer YLR275w-S2
caacgataactgaatcacctcttaagaatagtttacttatgcataggccactagtggatctg

SEQ ID NO: 5 - DNA, 59 bp, Artificial Sequence
  Description of Artificial Sequence primer YLR276c-S1
cttcaacgggtctactttaccattctttggcttactgactcagctgaagcttcgtacgc

SEQ ID NO: 6 - DNA, 62 bp, Artificial Sequence
  Description of Artificial Sequence primer YLR276c-S2
agctatgagaaaaagtctgtggaaggcgcttatattgacggcataggccactagtggatctg

SEQ ID NO: 7 - DNA, 59 bp, Artificial Sequence
  Description of Artificial Sequence primer YLR317w-S1
ctgccatcttctgccaccactttgtccttctttcttgatgcagctgaagcttcgtacgc

SEQ ID NO: 8 - DNA, 59 bp, Artificial Sequence
  Description of Artificial Sequence primer YLR359w-S1
ggctattgctgagaaggaattgggcttaactgttgttacacagctgaagcttcgtacgc

SEQ ID NO: 9 - DNA, 62 bp, Artificial Sequence
  Description of Artificial Sequence primer YLR359w-S2
aacttgacttgttcatcgtttaggtacttttggaaaggttgcataggccactagtggatctg

SEQ ID NO: 10 - DNA, 59 bp, Artificial Sequence
  Description of Artificial Sequence primer YLR373c-S1
acacacaggtacagagtgctgaaagaggattggtgttgcccagctgaagcttcgtacgc

SEQ ID NO: 11 - DNA, 62 bp, Artificial Sequence
  Description of Artificial Sequence primer YLR373c-S2
caaacagactttgttcctttgtatgtcctatggaagatacgcataggccactagtggatctg

SEQ ID NO: 12 - DNA, 59 bp, Artificial Sequence
  Description of Artificial Sequence primer YLR424w-S1
gacatgacatacactaatgatgccttgaaaactagtagcgcagctgaagcttcgtacgc

SEQ ID NO: 13 - DNA, 62 bp, Artificial Sequence
  Description of Artificial Sequence primer YLR424w-S2
ataggtactttctagaggtcaagggcccataaataaattggcataggccactagtggatctg

SEQ ID NO: 14 - DNA, 59 bp, Artificial Sequence
  Description of Artificial Sequence primer YLR437c-S1
attgtgcaagtctgttaaagtcttctcttggatccattaacagctgaagcttcgtacgc

SEQ ID NO: 15 - DNA, 62 bp, Artificial Sequence
  Description of Artificial Sequence primer YLR437c-S2
catcacacactaatacaggaacaaacaagacttaatggacgcataggccactagtggatctg

SEQ ID NO: 16 - DNA, 59 bp, Artificial Sequence
  Description of Artificial Sequence primer YLR440c-S1
ttgccaagaaaattgcagtaaaaatgttggaagagcaactcagctgaagcttcgtacgc

SEQ ID NO: 17 - DNA, 62 bp, Artificial Sequence
  Description of Artificial Sequence primer YLR440c-S2
gctccaattctagtgtgctccattgcgatgtaacaatttcgcataggccactagtggatctg

SEQ ID NO: 18 - DNA, 59 bp, Artificial Sequence
  Description of Artificial Sequence primer YFL024c-S1
tgatgaatttttctgggttatagaagagttctgtttcgctcagctgaagcttcgtacgc

SEQ ID NO: 19 - DNA, 62 bp, Artificial Sequence
  Description of Artificial Sequence primer YML049c-S2
actcctgcatcggacacttcgtcgatctggaagcagggtcgcataggccactagtggatctg

SEQ ID NO: 20 - DNA, 62 bp, Artificial Sequence
  Description of Artificial Sequence primer YFL024c-S2
acaccttcaaacgctatagagatcaatgacggttcgcatagcataggccactagtggatctg

SEQ ID NO: 21 - DNA, 59 bp, Artificial Sequence
  Description of Artificial Sequence primer YFR003c-S1
tgtggaagaggttcccgcagttttgcagcttcgagcaactcagctgaagcttcgtacgc

SEQ ID NO: 22 - DNA, 62 bp, Artificial Sequence
  Description of Artificial Sequence primer YFR003c-S2
atcttctttgtctacgttcgttaaagtcaagatccttctcgcataggccactagtggatctg

SEQ ID NO: 23 - DNA, 59 bp, Artificial Sequence
  Description of Artificial Sequence primer YFR027w-S1
aatgaaagctaggaaatcgcagagaaaagcgggcagtaaacagctgaagcttcgtacgc

SEQ ID NO: 24 - DNA, 62 bp, Artificial Sequence
  Description of Artificial Sequence primer YFR027w-S2
aatttggttgcgatacccaacttccttgctgtcctgcacagcataggccactagtggatctg

SEQ ID NO: 25 - DNA, 59 bp, Artificial Sequence
  Description of Artificial Sequence primer YFR042w-S1
agtttgcaccaatggcaatatgcctgtgataaagataaggcagctgaagcttcgtacgc

SEQ ID NO: 26 - DNA, 62 bp, Artificial Sequence
  Description of Artificial Sequence primer YFR042w-S2
catggaagttatttggttgcttagattccacgggttcaaagcataggccactagtggatctg

SEQ ID NO: 27 - DNA, 59 bp, Artificial Sequence
  Description of Artificial Sequence primer YIL109c-S1
tgtctcatcacaagaaacgtgtttacccacaagctcagctcagctgaagcttcgtacgc

SEQ ID NO: 28 - DNA, 59 bp, Artificial Sequence
  Description of Artificial Sequence primer YML077w-S1
atggggatatattcattttggatctttgataggcattgtacagctgaagcttcgtacgc

SEQ ID NO: 29 - DNA, 62 bp, Artificial Sequence
  Description of Artificial Sequence primer YIL109c-S2
tcatgatttgtaagaattctctgtaactttcgttattcaagcataggccactagtggatctg

SEQ ID NO: 30 - DNA, 59 bp, Artificial Sequence
  Description of Artificial Sequence primer YIL091c-S1
agtgacagttctgtgagggaaaagaatgataatttccgtgcagctgaagcttcgtacgc

SEQ ID NO: 31 - DNA, 62 bp, Artificial Sequence
  Description of Artificial Sequence primer YIL091c-S2
cattgtaaaattcaggattgtttggaggcttataaaaaacgcataggccactagtggatctg

SEQ ID NO: 32 - DNA, 59 bp, Artificial Sequence
  Description of Artificial Sequence primer YIL083c-S1
acctctacccgtgctcaacagacctcaaattcatacgtctcagctgaagcttcgtacgc

SEQ ID NO: 33 - DNA, 62 bp, Artificial Sequence
  Description of Artificial Sequence primer YIL083c-S2
cgatgacttctgggattatcatctcttcaatgatatggtggcataggccactagtggatctg

SEQ ID NO: 34 - DNA, 59 bp, Artificial Sequence
  Description of Artificial Sequence primer YIL019w-S1
ttcaaagaaaagcttttgaaagtcagttcggatctttagacagctgaagcttcgtacgc

SEQ ID NO: 35 - DNA, 62 bp, Artificial Sequence
  Description of Artificial Sequence primer YIL019w-S2
tagcgacgggatttctttttgccattaaatttaccactccgcataggccactagtggatctg

SEQ ID NO: 36 - DNA, 59 bp, Artificial Sequence
  Description of Artificial Sequence primer YIL104c-S1
tccccttactatttaagattaagatttcctcacgaattaacagctgaagcttcgtacgc

SEQ ID NO: 37 - DNA, 62 bp, Artificial Sequence
  Description of Artificial Sequence primer YIL104c-S2
cctgatacctgtaatgattgcaatcttgatgagagagctggcataggccactagtggatctg

SEQ ID NO: 38 - DNA, 59 bp, Artificial Sequence
  Description of Artificial Sequence primer YIR010w-S1
atgagtctggaacccacacaaacggtctccggtacgccgccagctgaagcttcgtacgc

SEQ ID NO: 39 - DNA, 62 bp, Artificial Sequence
  Description of Artificial Sequence primer YML077w-S2
ttctattggtgatctttcttgtcccttgacctctcatttcgcataggccactagtggatctg

SEQ ID NO: 40 - DNA, 62 bp, Artificial Sequence
  Description of Artificial Sequence primer YIR010w-S2
accttagctcatcgacttttgtttctaactttgtctcaacgcataggccactagtggatctg

SEQ ID NO: 41 - DNA, 59 bp, Artificial Sequence
  Description of Artificial Sequence primer YIR015w-S1
gcgaaccaggaccatttccatagattaaactacctctacccagctgaagcttcgtacgc

SEQ ID NO: 42 - DNA, 62 bp, Artificial Sequence
  Description of Artificial Sequence primer YIR015w-S2
atttccagtttttttggggtccataacaaccgatggcattgcataggccactagtggatctg

SEQ ID NO: 43 - DNA, 59 bp, Artificial Sequence
  Description of Artificial Sequence primer YPL233w-S1
atgtcacaaggtcagtccaaaaaactggacgtaactgttgcagctgaagcttcgtacgc

SEQ ID NO: 44 - DNA, 62 bp, Artificial Sequence
  Description of Artificial Sequence  primer YPL233w-S2
caatcctcctccaggaagtccattaagcgcttgaccttttgcataggccactagtggatctg

SEQ ID NO: 45 - DNA, 59 bp, Artificial Sequence
  Description of Artificial Sequence primer YPL146c-S1
tccaactaatctaaccaagaaaccatctcaatacaaacagcagctgaagcttcgtacgc

SEQ ID NO: 46 - DNA, 62 bp, Artificial Sequence
  Description of Artificial Sequence primer YPL146c-S2
tttgaagtccttatgtgtccacttttcagtgattttctgcgcataggccactagtggatctg

SEQ ID NO: 47 - DNA, 59 bp, Artificial Sequence
  Description of Artificial Sequence primer YPL126w-S1
atgacgcaatccctaggtatcgaacagtataaactgtcagcagctgaagcttcgtacgc

SEQ ID NO: 48 - DNA, 62 bp, Artificial Sequence
  Description of Artificial Sequence primer YPL126w-S2
tatgttaatactttcatcacacgatcaaaaaatgtatccagcataggccactagtggatctg

SEQ ID NO: 49 - DNA, 59 bp, Artificial Sequence
  Description of Artificial Sequence primer YPL093w-S1
caagattacaagaatcagagcgttctatatgcgtaaagttcagctgaagcttcgtacgc

SEQ ID NO: 50 - DNA, 59 bp, Artificial Sequence
  Description of Artificial Sequence primer YML093w-S1
gctaacttaaatatggcaaaaaagaaatctaagagcagatcagctgaagcttcgtacgc

SEQ ID NO: 51 - DNA, 62 bp, Artificial Sequence
  Description of Artificial Sequence primer YPL093w-S2
cggaaatctgtcttaccgacaccacgcttaccactgaatagcataggccactagtggatctg

SEQ ID NO: 52 - DNA, 59 bp, Artificial Sequence
  Description of Artificial Sequence primer YPL063w-S1
ttcaagcatccagaagacttttaacaagttattcaagtttcagctgaagcttcgtacgc

SEQ ID NO: 53 - DNA, 62 bp, Artificial Sequence
  Description of Artificial Sequence primer YPL063w-S2
ggattcagcaatcttcttctttttcttttcctcttcaaatgcataggccactagtggatctg

SEQ ID NO: 54 - DNA, 59 bp, Artificial Sequence
  Description of Artificial Sequence primer YPL024w-S1
atgtctttttcatctatcttatcacaggatatcacagatgcagctgaagcttcgtacgc

SEQ ID NO: 55 - DNA, 62 bp, Artificial Sequence
  Description of Artificial Sequence primer YPL024w-S2
acttgtgagtccttcaatatgaaaacgcccctattgaacagcataggccactagtggatctg

SEQ ID NO: 56 - DNA, 59 bp, Artificial Sequence
  Description of Artificial Sequence primer YPL020c-S1
tcagttgaagtagataagcaccggaacacactacagtatccagctgaagcttcgtacgc

SEQ ID NO: 57 - DNA, 62 bp, Artificial Sequence
  Description of Artificial Sequence primer YPL020c-S2
tcggttaaaatcaaatgggcaataaatcttctcatcctaagcataggccactagtggatctg

SEQ ID NO: 58 - DNA, 59 bp, Artificial Sequence
  Description of Artificial Sequence primer YPL012w-S1
atggatcaagacaaagttgcttttcttttagagctggaggcagctgaagcttcgtacgc

SEQ ID NO: 59 - DNA, 62 bp, Artificial Sequence
  Description of Artificial Sequence primer YPL012w-S2
atttgaactttggacctttcttattatgtttgccaatcttgcataggccactagtggatctg

SEQ ID NO: 60 - DNA, 59 bp, Artificial Sequence
  Description of Artificial Sequence primer YPL007c-S1
cccgttgtttgctcacgggacatatctttacatgcgttgtcagctgaagcttcgtacgc

SEQ ID NO: 61 - DNA, 62 bp, Artificial Sequence
  Description of Artificial Sequence primer YML093w-S2
caaaggatcaataacttggcctggcttagtcatgattctcgcataggccactagtggatctg

SEQ ID NO: 62 - DNA, 62 bp, Artificial Sequence
  Description of Artificial Sequence primer YPL007c-S2
ggacttattggtagatagaaaggaatttgaggattggaaggcataggccactagtggatctg

SEQ ID NO: 63 - DNA, 59 bp, Artificial Sequence
  Description of Artificial Sequence primer YPR048w-S1
atgtcatcgagcaagaaaatcgtcatcctctatggatcggcagctgaagcttcgtacgc

SEQ ID NO: 64 - DNA, 62 bp, Artificial Sequence
  Description of Artificial Sequence primer YPR048w-S2
aattagttatttcctcacctaatctccataagtagtcttggcataggccactagtggatctg

SEQ ID NO: 65 - DNA, 59 bp, Artificial Sequence
  Description of Artificial Sequence primer YPR072w-S1
tgtctcaaagaaagctacaacaggatatcgataagcttttcagctgaagcttcgtacgc

SEQ ID NO: 66 - DNA, 62 bp, Artificial Sequence
  Description of Artificial Sequence primer YPR072w-S2
agatttgcccattcctggtggtaacttttcgatttctttagcataggccactagtggatctg

SEQ ID NO: 67 - DNA, 59 bp, Artificial Sequence
  Description of Artificial Sequence primer YPR082c-S1
cttcgattgctgaaagagtaaggaactttgcagttatttacagctgaagcttcgtacgc

SEQ ID NO: 68 - DNA, 62 bp, Artificial Sequence
  Description of Artificial Sequence primer YPR082c-S2
caataaagttcaacttgttgttgttccctgtaccaaaatcgcataggccactagtggatctg

SEQ ID NO: 69 - DNA, 59 bp, Artificial Sequence
  Description of Artificial Sequence primer YPR085c-S1
ctgtacattctttcgaaagactccatgctgcgaatttttgcagctgaagcttcgtacgc

SEQ ID NO: 70 - DNA, 62 bp, Artificial Sequence
  Description of Artificial Sequence primer YPR085c-S2
tcccactttatagttatgggatttcgagctggattcggtagcataggccactagtggatctg

SEQ ID NO: 71 - DNA, 59 bp, Artificial Sequence
  Description of Artificial Sequence primer YML114c-S1
aacgtgtaattgagggactcataaagggcaatgacttccacagctgaagcttcgtacgc

SEQ ID NO: 72 - DNA, 59 bp, Artificial Sequence
  Description of Artificial Sequence primer YPR105c-S1
agctcgatcatcgagggccaattgtctaaaaatctagcaacagctgaagcttcgtacgc

SEQ ID NO: 73 - DNA, 62 bp, Artificial Sequence
  Description of Artificial Sequence primer YPR105c-S2
ctgtgttctatcaatcttcatatttctagctttaattcttgcataggccactagtggatctg

SEQ ID NO: 74 - DNA, 59 bp, Artificial Sequence
  Description of Artificial Sequence primer YPR112c-S1
cattgtcaagggtttgcccgtctatctaacagatgataatcagctgaagcttcgtacgc

SEQ ID NO: 75 - DNA, 59 bp, Artificial Sequence
  Description of Artificial Sequence primer YPR137c-S1
atgtcagatgttacccaacagaaaaagaggaaaagatccacagctgaagcttcgtacgc

SEQ ID NO: 76 - DNA, 62 bp, Artificial Sequence
  Description of Artificial Sequence primer YPR112c-S2
gaaaccttcgttttcttcatcatccacatccagtttctttgcataggccactagtggatctg

SEQ ID NO: 77 - DNA, 59 bp, Artificial Sequence
  Description of Artificial Sequence primer YPR143w-S1
atgggctccaagcacagagtagacactaaggataagaaaacagctgaagcttcgtacgc

SEQ ID NO: 78 - DNA, 62 bp, Artificial Sequence
  Description of Artificial Sequence primer YPR143w-S2
ttcattgtcgcttcctgcggcagctttaactaaatccaaagcataggccactagtggatctg

SEQ ID NO: 79 - DNA, 59 bp, Artificial Sequence
  Description of Artificial Sequence primer YPR144c-S1
ttccagaaaatgttactcaattggaagaagatgagacagacagctgaagcttcgtacgc

SEQ ID NO: 80 - DNA, 62 bp, Artificial Sequence
  Description of Artificial Sequence primer YPR144c-S2
ccatgctaccccaggcaagtagacgttaccttgggatgacgcataggccactagtggatctg

SEQ ID NO: 81 - DNA, 62 bp, Artificial Sequence
  Description of Artificial Sequence primer YML114c-S2
gacttgtagtagcatcgatattggttgtgttatgtgctacgcataggccactagtggatctg

SEQ ID NO: 82 - DNA, 59 bp, Artificial Sequence
  Description of Artificial Sequence primer YPR169w-S1
ttttacatcctgaactgcccattataataactggctttggcagctgaagcttcgtacgc

SEQ ID NO: 83 - DNA, 62 bp, Artificial Sequence
  Description of Artificial Sequence primer YPR169w-S2
cttcttgatcccatgctcatacaggtccttttttttgttggcataggccactagtggatctg

SEQ ID NO: 84 - DNA, 59 bp, Artificial Sequence
  Description of Artificial Sequence primer YML127w-S1
ccgctaaatggtactccagtaagcgaggcacccgccacaacagctgaagcttcgtacgc

SEQ ID NO: 85 - DNA, 62 bp, Artificial Sequence
  Description of Artificial Sequence primer YML127w-S2
ataaccccgacgtgttttccatgtattcagacaatgctaagcataggccactagtggatctg

SEQ ID NO: 86 - DNA, 59 bp, Artificial Sequence
  Description of Artificial Sequence primer YMR032w-S1
ctacagttatgaagcttgtttttgggacccaaacgacaatcagctgaagcttcgtacgc

SEQ ID NO: 87 - DNA, 62 bp, Artificial Sequence
  Description of Artificial Sequence primer YMR032w-S2
cagaaaactagtaaaattgatatacatcgagatcaaagacgcataggccactagtggatctg

SEQ ID NO: 88 - DNA, 59 bp, Artificial Sequence
  Description of Artificial Sequence primer YMR093w-S1
atgtcgactgctaggcctagaataatcacttcgaaggctccagctgaagcttcgtacgc

SEQ ID NO: 89 - DNA, 62 bp, Artificial Sequence
  Description of Artificial Sequence primer YMR093w-S2
aagcaccaattcagtagcggctctaatgtagattcatctcgcataggccactagtggatctg

SEQ ID NO: 90 - DNA, 59 bp, Artificial Sequence
  Description of Artificial Sequence primer YMR131c-S1
ctttaacttccttttgccagtgaacaaacaataattgtggcagctgaagcttcgtacgc

SEQ ID NO: 91 - DNA, 62 bp, Artificial Sequence
  Description of Artificial Sequence primer YMR131c-S2
ggtctatcgaggtcaacgaggaacaagatagagtggtctcgcataggccactagtggatctg

SEQ ID NO: 92 - DNA, 59 bp, Artificial Sequence
  Description of Artificial Sequence primer YMR185w-S1
atcaacatacacgatatattgaatacaagaccgaagctcacagctgaagcttcgtacgc

SEQ ID NO: 93 - DNA, 62 bp, Artificial Sequence
  Description of Artificial Sequence primer YMR185w-S2
gtaatgggttataaactatctagtacggttaaaagcttgtgcataggccactagtggatctg

SEQ ID NO: 94 - DNA, 59 bp, Artificial Sequence
  Description of Artificial Sequence primer YMR212c-S1
cctcttgaacttaaagaatgtaaatcttcatttgcgtcttcagctgaagcttcgtacgc

SEQ ID NO: 95 - DNA, 59 bp, Artificial Sequence
  Description of Artificial Sequence primer YDR499w-S1
atgagacgagaaacggtgggtgaattttcttcagatgacgcagctgaagcttcgtacgc

SEQ ID NO: 96 - DNA, 62 bp, Artificial Sequence
  Description of Artificial Sequence primer YMR212c-S2
cggatgatgttcacaccaaaacatcagaaactggtcaatcgcataggccactagtggatctg

SEQ ID NO: 97 - DNA, 59 bp, Artificial Sequence
  Description of Artificial Sequence primer YMR213w-S1
atacgtgaaaggcggtgtatggaccaatgtggaggatcagcagctgaagcttcgtacgc

SEQ ID NO: 98 - DNA, 62 bp, Artificial Sequence
  Description of Artificial Sequence primer YMR213w-S2
gctgtaactgttcaatagactccacttttgattggatcgagcataggccactagtggatctg

SEQ ID NO: 99 - DNA, 59 bp, Artificial Sequence
  Description of Artificial Sequence primer YMR218c-S1
gactcaaatgcattagagtgatcaactctacaacttttaccagctgaagcttcgtacgc

SEQ ID NO: 100 - DNA, 62 bp, Artificial Sequence
  Description of Artificial Sequence primer YMR218c-S2
gaaggcatttgacggaactgtacgaacggttaacaggcttgcataggccactagtggatctg

SEQ ID NO: 101 - DNA, 59 bp, Artificial Sequence
  Description of Artificial Sequence primer YMR281w-S1
ctgaagaaaagttaaatgaagatgttgaggcgtacaaaggcagctgaagcttcgtacgc

SEQ ID NO: 102 - DNA, 62 bp, Artificial Sequence
  Description of Artificial Sequence primer YMR281w-S2
agtacgtattgtgcatgtgtattcataagtgaaagcttgtgcataggccactagtggatctg

SEQ ID NO: 103 - DNA, 59 bp, Artificial Sequence
  Description of Artificial Sequence YMR288w-S1
gaaaacctgcagaaagaagctgcacgtattggtgagaacgcagctgaagcttcgtacgc

SEQ ID NO: 104 - DNA, 62 bp, Artificial Sequence
  Description of Artificial Sequence primer YMR288w-S2
ccaaaccttctaaaatacgcataatagcatgtggtgaagtgcataggccactagtggatctg

SEQ ID NO: 105 - DNA, 59 bp, Artificial Sequence
  Description of Artificial Sequence primer YMR290c-S1
tgagttttacgtcttttggtatttggcgtttttccactggcagctgaagcttcgtacgc

SEQ ID NO: 106 - DNA, 59 bp, Artificial Sequence
  Description of Artificial Sequence primer YML049c-S1
aattcctgctcattcaaggaaagtctcaggaaattttcaccagctgaagcttcgtacgc

SEQ ID NO: 107 - DNA, 62 bp, Artificial Sequence
  Description of Artificial Sequence primer YMR290c-S2
gataagctgagcaatattaacaggagaagtatggctacccgcataggccactagtggatctg

SEQ ID NO: 108 - DNA, 59 bp, Artificial Sequence
  Description of Artificial Sequence primer YMR211w-S1
agagagcaaaccatttgactactcaattcttcaatatacacagctgaagcttcgtacgc

SEQ ID NO: 109 - DNA, 62 bp, Artificial Sequence
  Description of Artificial Sequence primer YMR211w-S2
atttcaatcatcttactccgtgaatcaggttcggaatgatgcataggccactagtggatctg

SEQ ID NO: 110 - DNA, 59 bp, Artificial Sequence
  Description of Artificial Sequence primer YDR196c-S1
atgcttatgatcaaattgtgttatacttcaaggacaaaatcagctgaagcttcgtacgc

SEQ ID NO: 111 - DNA, 62 bp, Artificial Sequence
  Description of Artificial Sequence primer YDR196c-S2
tttcaatctgttcgtataagtcaaccaatgtgctgttattgcataggccactagtggatctg

SEQ ID NO: 112 - DNA, 59 bp, Artificial Sequence
  Description of Artificial Sequence primer YDR299w-S1
atggaaaaatcactagcggatcaaatttccgatatcgccacagctgaagcttcgtacgc

SEQ ID NO: 113 - DNA, 62 bp, Artificial Sequence
  Description of Artificial Sequence primer YDR299w-S2
caaagatttggatatcatcgtttttaacagcctctaattcgcataggccactagtggatctg

SEQ ID NO: 114 - DNA, 59 bp, Artificial Sequence
  Description of Artificial Sequence primer YDR365c-S1
ctggagagaacccaaagaaggaaggtgtagatgctaggttcagctgaagcttcgtacgc

SEQ ID NO: 115 - DNA, 62 bp, Artificial Sequence
  Description of Artificial Sequence primer YDR365c-S2
ttagtatgctttttattaacagatttcaacttgcttttctgcataggccactagtggatctg

SEQ ID NO: 116 - DNA, 59 bp, Artificial Sequence
  Description of Artificial Sequence primer YDR396w-S1
cagatacactattgtggtgtaatctggaccttgactgtctcagctgaagcttcgtacgc

SEQ ID NO: 117 - DNA, 59 bp, Artificial Sequence
  Description of Artificial Sequence primer YLR222c-S1
ctctcaacggtagtaagccatactacgtacaatatggatccagctgaagcttcgtacgc

SEQ ID NO: 118 - DNA, 62 bp, Artificial Sequence
  Description of Artificial Sequence primer YDR396w-S2
tagagaaaacactgaatgatcttagcgaccgtacaaaagagcataggccactagtggatctg

SEQ ID NO: 119 - DNA, 59 bp, Artificial Sequence
  Description of Artificial Sequence primer YDR407c-S1
ttcttaagcatttcccaagctatgttggcccatctaagatcagctgaagcttcgtacgc

SEQ ID NO: 120 - DNA, 62 bp, Artificial Sequence
  Description of Artificial Sequence primer YDR407c-S2
aataacagacaagataacgttttcagagtcgaactggattgcataggccactagtggatctg

SEQ ID NO: 121 - DNA, 59 bp, Artificial Sequence
  Description of Artificial Sequence primer YDR416w-S1
acttacatggaaaagatatatcgagtattggaaagaggagcagctgaagcttcgtacgc

SEQ ID NO: 122 - DNA, 62 bp, Artificial Sequence
  Description of Artificial Sequence YDR416w-S2
tcaaatatctagttctatttcatctggattaatcgaatatgcataggccactagtggatctg

SEQ ID NO: 123 - DNA, 59 bp, Artificial Sequence
  Description of Artificial Sequence primer YDR449c-S1
cacatcaccgatttctaataatgtcgaagacaagatactacagctgaagcttcgtacgc

SEQ ID NO: 124 - DNA, 62 bp, Artificial Sequence
  Description of Artificial Sequence primer YDR449c-S2
ataattaaatctagaattttatacctaggatcatcttctggcataggccactagtggatctg

SEQ ID NO: 125 - DNA, 59 bp, Artificial Sequence
  Description of Artificial Sequence primer YDR141c-S1
ttcgtaatctttgaattctgcgatttcatctaccagcgcgcagctgaagcttcgtacgc

SEQ ID NO: 126 - DNA, 62 bp, Artificial Sequence
  Description of Artificial Sequence primer YDR141c-S2
cactaaagccccttacaattgactcaaataataaacaactgcataggccactagtggatctg

SEQ ID NO: 127 - DNA, 59 bp, Artificial Sequence
  Description of Artificial Sequence primer YDR324c-S1
aagaagcctgaaaatacgaaacaaaccggtgaagatgacccagctgaagcttcgtacgc

SEQ ID NO: 128 - DNA, 62 bp, Artificial Sequence
  Description of Artificial Sequence primer YMR049c-S2
tctgttctaacataactaggtcaatgatggctaagaacaagcataggccactagtggatctg

SEQ ID NO: 129 - DNA, 62 bp, Artificial Sequence
  Description of Artificial Sequence primerYDR324c-S2
aaacactaactttggttgaataaacgccttttgtttggaggcataggccactagtggatctg

SEQ ID NO: 130 - DNA, 59 bp, Artificial Sequence
  Description of Artificial Sequence primer YDR325w-S1
gacattaatacgaaaatctttaactcagttgctgaagtatcagctgaagcttcgtacgc

SEQ ID NO: 131 - DNA, 62 bp, Artificial Sequence
  Description of Artificial Sequence primer YDR325w-S2
acctcgctgaaagactctgaatccttatcttcttcatctagcataggccactagtggatctg

SEQ ID NO: 132 - DNA, 59 bp, Artificial Sequence
  Description of Artificial Sequence primer YDR398w-S1
atggattctcctgttctacagtccgcttatgacccatcagcagctgaagcttcgtacgc

SEQ ID NO: 133 - DNA, 62 bp, Artificial Sequence
  Description of Artificial Sequence primer YDR398w-S2
aacgtcactatatccggcttcctcctcgccgtcgctctgcgcataggccactagtggatctg

SEQ ID NO: 134 - DNA, 59 bp, Artificial Sequence
  Description of Artificial Sequence primer YDR246w-S1
atggccatcgaaacaatacttgtaataaacaaatcaggcgcagctgaagcttcgtacgc

SEQ ID NO: 135 - DNA, 62 bp, Artificial Sequence
  Description of Artificial Sequence primer YDR246w-S2
aacaggttagatcttataggcatttccattgagtaagatggcataggccactagtggatctg

SEQ ID NO: 136 - DNA, 59 bp, Artificial Sequence
  Description of Artificial Sequence primer YDR236c-S1
ctaaaatattgaacttgaccctggccccataaaaatcattcagctgaagcttcgtacgc

SEQ ID NO: 137 - DNA, 62 bp, Artificial Sequence
  Description of Artificial Sequence primer YDR236c-S2
ttgaagtgttgatgtttacgtggactatttatgtttcgttgcataggccactagtggatctg

SEQ ID NO: 138 - DNA, 62 bp, Artificial Sequence
  Description of Artificial Sequence primer YDR361c-S2
ttaccaagtggaaatttctgtttccaattcatcgatacttgcataggccactagtggatctg

SEQ ID NO: 139 - DNA, 59 bp, Artificial Sequence
  Description of Artificial Sequence primer YMR134w-S1
gcaaagtgtggtatagaaaaagaaccaaaggccggtatgtcagctgaagcttcgtacgc

SEQ ID NO: 140 - DNA, 59 bp, Artificial Sequence
  Description of Artificial Sequence primer YDR361c-S1
ggttcaagctatcaaattaaatgatttaaaaaataggaagcagctgaagcttcgtacgc

SEQ ID NO: 141 - DNA, 59 bp, Artificial Sequence
  Description of Artificial Sequence primer YDR367w-S1
atctgcgtactttatacaatcgataccatttccacttgttcagctgaagcttcgtacgc

SEQ ID NO: 142 - DNA, 62 bp, Artificial Sequence
  Description of Artificial Sequence primer YDR367w-S2
gttttgttctacgtcatccctatcaactaaatatttgggggcataggccactagtggatctg

SEQ ID NO: 143 - DNA, 59 bp, Artificial Sequence
  Description of Artificial Sequence YDR339c-S1
tatgggtaaagctaagaaaacaagaaagtttggcctcgtacagctgaagcttcgtacgc

SEQ ID NO: 144 - DNA, 62 bp, Artificial Sequence
  Description of Artificial Sequence primer YDR339c-S2
taaaagacatctggcaatttttcaatgacgtatgcgtgacgcataggccactagtggatctg

SEQ ID NO: 145 - DNA, 59 bp, Artificial Sequence
  Description of Artificial Sequence primer YDR413c-S1
ttctttggtttattcttcgttcatttttggtcaaatatctcagctgaagcttcgtacgc

SEQ ID NO: 146 - DNA, 62 bp, Artificial Sequence
  Description of Artificial Sequence primer YDR413c-S2
acaaaagaaagcacaagagtttattaaggagcaggaaagggcataggccactagtggatctg

SEQ ID NO: 147 - DNA, 59 bp, Artificial Sequence
  Description of Artificial Sequence primer YDR429c-S1
tctagatctatcattacatacaagattgaagacggtgtcacagctgaagcttcgtacgc

SEQ ID NO: 148 - DNA, 62 bp, Artificial Sequence
  Description of Artificial Sequence primer YDR429c-S2
tttctttgtttctaacgacagaaactcttggaatgggtgcgcataggccactagtggatctg

SEQ ID NO: 149 - DNA, 59 bp, Artificial Sequence
  Description of Artificial Sequence primer YDR468c-S1
gtcacaatactgctggtgatgacgatcaagaggaggaaatcagctgaagcttcgtacgc

SEQ ID NO: 150 - DNA, 62 bp, Artificial Sequence
  Description of Artificial Sequence primer YMR134w-S2
tgtgtgtgtgcctacctgcatgtatgcatttagcaattgagcataggccactagtggatctg

SEQ ID NO: 151 - DNA, 62 bp, Artificial Sequence
  Description of Artificial Sequence primer YDR468c-S2
caagacgacaataagaagtcctatacaacaatcgtcgtatgcataggccactagtggatctg

SEQ ID NO: 152 - DNA, 59 bp, Artificial Sequence
  Description of Artificial Sequence primer YDR489w-S1
actacccacagagatgcaaatacaatagtgggttcgtcctcagctgaagcttcgtacgc

SEQ ID NO: 153 - DNA, 62 bp, Artificial Sequence
  Description of Artificial Sequence primer YDR527w-S2
ccccaccgccttgtttccataaccaaagtgcatcaatagcgcataggccactagtggatctg

SEQ ID NO: 154 - DNA, 59 bp, Artificial Sequence
  Description of Artificial Sequence primer YDR288w-S1
atgagttctatagataatgacagcgatgtggatttaacagcagctgaagcttcgtacgc

SEQ ID NO: 155 - DNA, 62 bp, Artificial Sequence
  Description of Artificial Sequence primer YDR288w-S2
gcccatgatttcttgcaccaatttttcaagagactctagtgcataggccactagtggatctg

SEQ ID NO: 156 - DNA, 59 bp, Artificial Sequence
  Description of Artificial Sequence primer YDR201w-S1
cccatgtctggactattcagagcatcatcgtcatccataccagctgaagcttcgtacgc

SEQ ID NO: 157 - DNA, 62 bp, Artificial Sequence
  Description of Artificial Sequence primer YDR201w-S2
aaaagggttttccgtttagttcccgaatatgatgttgaaagcataggccactagtggatctg

SEQ ID NO: 158 - DNA, 59 bp, Artificial Sequence
  Description of Artificial Sequence primer YDR434w-S1
atgtccaatgcaaatctaagaaaatgggttggtttttgctcagctgaagcttcgtacgc

SEQ ID NO: 159 - DNA, 59 bp, Artificial Sequence
  Description of Artificial Sequence primer YML023c-S1
cacgcaatggtgcacattattttgttgaactcactgagaacagctgaagcttcgtacgc

SEQ ID NO: 160 - DNA, 62 bp, Artificial Sequence
  Description of Artificial Sequence primer YDR434w-S2
taaaggtaaatacacagctatcatgtgctcttgtgggaaggcataggccactagtggatctg

SEQ ID NO: 161 - DNA, 59 bp, Artificial Sequence
  Description of Artificial Sequence primer YDR181c-S1
aggataaacccaaatgctggacatctaaggaaatctaagtcagctgaagcttcgtacgc

SEQ ID NO: 162 - DNA, 62 bp, Artificial Sequence
  Description of Artificial Sequence primer YDR181c-S2
tagttgggtttgaatcgttatcacgggagaacattgctttgcataggccactagtggatctg

SEQ ID NO: 163 - DNA, 59 bp, Artificial Sequence
  Description of Artificial Sequence primer YDR531w-S1
atgccgcgaattactcaagagatatcttacaattgcgattcagctgaagcttcgtacgc

SEQ ID NO: 164 - DNA, 62 bp, Artificial Sequence
  Description of Artificial Sequence primerYDR531w-S2
aaataagctatttgcccaatattgttggagatggcgaatagcataggccactagtggatctg

SEQ ID NO: 165 - DNA, 59 bp, Artificial Sequence
  Description of Artificial Sequence primer YLR186w-S1
ctagtcaccaagaagaaaacccgtaaaatcgtaggtcatgcagctgaagcttcgtacgc

SEQ ID NO: 166 - DNA, 62 bp, Artificial Sequence
  Description of Artificial Sequence primer YLR186w-S2
atacaaagaggatgccaagtagacttaaacactataaaatgcataggccactagtggatctg

SEQ ID NO: 167 - DNA, 59 bp, Artificial Sequence
  Description of Artificial Sequence primerYDR472w-S1
atgtctcaaagaataattcaaccaagcgcatctgaccaaccagctgaagcttcgtacgc

SEQ ID NO: 168 - DNA, 59 bp, Artificial Sequence
  Description of Artificial Sequence primer YLR215c-S1
ttacttattgatgtcctcacaagaatatacaacttttatacagctgaagcttcgtacgc

SEQ ID NO: 169 - DNA, 62 bp, Artificial Sequence
  Description of Artificial Sequence primer YLR215c-S2
agctctcggattgcttcaggatttaaactagcttctacgagcataggccactagtggatctg

SEQ ID NO: 170 - DNA, 59 bp, Artificial Sequence
  Description of Artificial Sequence primer YMR049c-S1
cagactattgattactttatgaccggttagtttctttagtcagctgaagcttcgtacgc

SEQ ID NO: 171 - DNA, 62 bp, Artificial Sequence
  Description of Artificial Sequence primer YLR243w-S2
gataatatggtttctatactgtcaggattattagattccagcataggccactagtggatctg

SEQ ID NO: 172 - DNA, 62 bp, Artificial Sequence
  Description of Artificial Sequence PRIMER YLR272c-S2
ggtaactgacttcgttactttatgagatgtccggctttaggcataggccactagtggatctg

SEQ ID NO: 173 - DNA, 62 bp, Artificial Sequence
  Description of Artificial Sequence primer YML023c-S2
attagttacttattctataattacacttttatcatgaacggcataggccactagtggatctg

SEQ ID NO: 174 - DNA, 62 bp, Artificial Sequence
  Description of Artificial Sequence primer YLR222c-S2
aatatgtaactttgttcaactaagttatcaacccttgtgagcataggccactagtggatctg

SEQ ID NO: 175 - DNA, 62 bp, Artificial Sequence
  Description of Artificial Sequence primer YDR499c-S2
cgtactttacttgcattattctccccgttcttttattcaagcataggccactagtggatgtg

SEQ ID NO: 176 - DNA, 62 bp, Artificial Sequence
  Description of Artificial Sequence primer YLR317w-S2
gaagtaaactaactagtaaagtaggctaattcgaaacgatgcataggccactagtggatctg

SEQ ID NO: 177 - DNA, 62 bp, Artificial Sequence
  Description of Artificial Sequence primer YDR472w-S2
agccaaatctcaaaccttccctgtcaagcacttgcctgtcgcataggccactagtggatgtg

SEQ ID NO: 178 - DNA, 62 bp, Artificial Sequence
  Description of Artificial Sequence primer YDR489w-S2
agtcgggctcatctatcatgtttacgctaccttctgtatcgcataggccactagtggatctg

SEQ ID NO: 179 - DNA, 59 bp, Artificial Sequence
  Description of Artificial Sequence primer YDR527w-S1
atggacttactgggcgatatagtggagaaagatacatctgcagctgaagcttcgtacgc

SEQ ID NO: 180 - DNA, 62 bp, Artificial Sequence
  Description of Artificial Sequence primer YPR137w-S2
aaaagcctgtttggtcaatgacagctgaatatataccattgcataggccactagtggatctg

(19) United States
(12) Patent Application Publication
(10) Pub. No.: US 2004/0229276 A1
(43) Pub. Date: Nov. 18, 2004

(54) Method for screening antimycotic substances substances using essential genes from S. cerevisiae

(75) Inventors: Anita Diu-Hercend, Charento Le Pont (FR); Karl-Dieter Entian, Oberursel (DE); Peter Koetter, Oberursel (DE)

Correspondence Address: Charles A. Muserlian, c/o Muserlian, Lucas and Mercanti, 475 Park Avenue South, New York, NY, 10016, US

(73) Assignee: Hoechst Marion Roussel

(21) Appl. No.: 10/872,929

(22) Filed: Jun. 21, 2004

Related U.S. Application Data
(63) Continuation of application No. 09/674,109, filed on Jun. 21, 2001.
Application No. 09/674,109 is a 371 of international application No. PCT/EP99/02722, filed on Apr. 22, 1999.

(30) Foreign Application Priority Data
Apr. 24, 1998 (EP) 98401007.4
Sep. 11, 1998 (EP) 98402254.1

(51) Int. Cl. C12Q 1/68; C12N 9/14

(52) U.S. Cl. 435/6

(57) ABSTRACT

The present invention concerns a method for the screening of antimycotic substances wherein an essential gene from mycetes or a functionally similar mycete gene, or the corresponding encoded protein, is used as target and wherein the essential gene is selected from the group consisting in YML114c, YLR186w, YLR215c, YLR222c, YLR243w, YLR272c, YLR275w, YLR276c, YLR317w, YLR359w, YLR373c, YLR424w, YLR437c, YLR440c, YML023c, YML049c, YML077w, YML093w, YML127w, YMR032w, YMR093w, YMR131c, YMR185w, YMR212c, YMR213w, YMR218c, YMR281w, YMR288w, YMR290c, YMR211w, YMR049c, YMR134w, YDR196c, YDR299w, YDR365c, YDR396w, YDR407c, YDR416w, YDR449c, YDR472w, YDR499w, YDR141c, YDR324c, YDR325w, YDR398w, YDR246w, YDR236c, YDR361c, YDR367w, YDR339c, YDR413c, YDR429c, YDR468c, YDR489w, YDR527w, YDR288w, YDR201w, YDR434w, YDR181c, YDR531w, YPL126w, YPL093w, YPL063w, YPL024w, YPL020c, YPL012w, YPL007c, YPL233w, YPLI46c, YIL091c, YIL083c, YIL019w, YIL109c, YIL104c, YFL024c, YFR003c, YFR027w, YFR042w, YIR010w, YIR015w, YPR048w, YPR072w, YPR082c, YPR085c, YPR105c, YPR112c, YPR137w, YPR143w, YPR144c and YPR169w.

The present invention relates to a method for screening for antimycotic substances in which essential genes from mycetes, particularly from Saccaromyces cerevisiae (S. cerevisiae) as well as functionally similar genes from other mycetes, or the corresponding encoded proteins, are used as targets.

The spectrum of known fungal infections stretches from fungal attack of skin or nails to potentially hazardous mycotic infections of the inner organs; Such infections and resulting diseases are known as mycosis.

Antimycotic substances (fungistatic or fungicidal) are used for treatment of mycosis. However, up to now, relatively few substances with pharmacological effects are known, such as Amphotericin B, Nystatin, Pimaricin, Griseofulvin, Clotrimazole, 5-fluoro-cytosine and Batraphene. The drug treatment of fungal infections is extremely difficult, in particular because both the host cells and the mycetes, are eukaryotic cells. Administration of drugs based on known antimycotic substances results therefore often in undesired side-effects, for example Amphotericin B has a nephrotoxic effect. Therefore, there is a strong need for pharmacologically efficient substances usable for the preparation of drugs, which are suitable for prophylactic treatments of immunodepressive states or for the treatment of an existing fungal infection. Furthermore, the substances should exhibit a specific spectrum of action in order to selectively inhibit the growth and proliferation of mycetes without affecting the treated host organism.

The aim of the present invention is to provide a method for the identification of antimycotic substances. An essential feature of this method is that essential genes from mycetes are used as targets for the screening.

The present invention thus concerns a method for screening antimycotic substances wherein an essential gene from mycetes or a functionally similar gene in another mycete, or the corresponding encoded protein, is used as target and wherein the essential gene is selected from the group consisting in YML114c, YLR186w, YLR215c, YLR222c, YLR243w, YLR272c, YLR275w, YLR276c, YLR317w, YLR359w, YLR373c, YLR424w, YLR437c, YLR440c, YML023c, YML049c, YML077w, YML093w, YML127w, YMR032w, YMR093w, YMR131c, YMR185w, YMR212c, YMR213w, YMR218c, YMR281w, YMR288w, YMR290c, YMR211w, YMR049c, YMR134w, YDR196c, YDR299w, YDR365c, YDR396w, YDR407c, YDR416w, YDR449c, YDR472w, YDR499w, YDR141c, YDR324c, YDR325w, YDR398w, YDR246w, YDR236c, YDR361c, YDR367w, YDR339c, YDR413c, YDR429c, YDR468c, YDR489w, YDR5:27w, YDR288w, YDR201w, YDR434w, YDR181c, YDR531w, YPL126w, YPL093w, YPL063w, YPL024w, YPL020c, YPL012w, YPL007c, YPL233w, YPL146c, YIL091c, YIL083c, YIL019w, YIL109c, YIL104c, YFL024c, YFR003c, YFR027w, YFR042w, YIR010w, YIR015w, YPR048w, YPR072w, YPR082c, YPR085c, YPR105c, YPR112c, YPR137w, YPR143w, YPR144c and YPR169w.

According to one embodiment of the method of the invention mycete cells which express the essential gene, or a functionally similar mycete gene, to a different level are incubated with the substance to be tested and the growth inhibiting effect of the substance is determined.

According to another embodiment, said target gene or the corresponding target gene encoded protein is contacted in vitro with the substance to be tested and the effect of the substance on the target is determined.

According to another embodiment, the screened substances inhibit partially or totally the functional expression of the essential genes or the functional activity of the encoded proteins.

According to another embodiment, the mycete species are selected from the group comprising Basidiomycetes, Ascomycetes and Hyphomycetes.

According to another embodiment of the method of the invention said functional similar genes are essential genes from Candida Spp., preferably Candida albicans, or from Aspergillus Spp., preferably from Aspergillus fumigatus.

According to a further embodiment of the above method said mycete cells are haploid S. cerevisiae cells.

According to a particular embodiment of the method of the invention the essential genes of S. cerevisiae are identified by integrating by homologous recombination a selection marker at the locus of the gene to be studied.

The present invention also concerns a method as described above wherein the functionally similar genes are identified by:

a) providing a S. cerevisiae mutant strain in which the gene of S. cerevisiae to be investigated is either integrative or extrachromosomal under the control of a regulated promoter,

b) culturing said mutant strain under growth conditions in which the regulated promoter is active,

c)transforming the mutant strain with a cDNA or genomic DNA that has been prepared from the heterologous mycete-species and that has been integrated into an appropriate vector,

d) altering the culture condition, so that the regulated promoter is switched off and only S. cerevisiae cells which contain a functionally similar gene can survive,

e)isolating and analyzing the cDNA or genomic DNA.

The invention thus discloses that in a first step, essential genes from S. cerevisiae are identified. The invention also discloses that, essential genes from other mycetes are identified starting from the identified essential genes in S. cerevisiae. In order to identify essential genes of S. cerevisiae, individual genomic genes are eliminated through homologous recombination. If the DNA segment thus eliminated concerns an essential gene, then the deletion is lethal for the S. cerevisiae cells in haploid form.

A method, wherein the studied S. cerevisiae gene is replaced by a marker gene can be used to generate the corresponding genomic deletion of S. cerevisiae and to determine the S. cerevisiae cells containing the deletion.

As a selection marker a dominant selection marker (e.g. kanamycin resistance gene) or an auxotrophic marker can for example be used. As an auxotrophic marker, it is possible to use genes coding for key enzymes of amino acid or nucleic base synthesis. For example, one can use as a selection marker the following genes from S. cerevisiae: gene encoding for the metabolic pathway of leucine (e.g. LEU2-gene), histidine (e.g. HIS3-gene) or tryptophan (e.g. TRP1 gene) or for the nucleic base metabolism of uracil (e.g. URA3-gene).

Auxotrophic S. cerevisiae strains can be used. These auxotrophic strains can only grow on nutritive media containing the corresponding amino acids or nucleotide bases. All laboratory S. cerevisiae strains, containing auxotrophic markers can for instance be used. When diploid S. cerevisiae strains are used, then the corresponding marker gene must be homozygously mutated. Strain CEN.PK2 or isogenic derivates thereof can be used.

Strains containing no suitable auxotrophic marker can also be used such as prototrophic S. cerevisiae strains. Then a dominant selection marker e.g. resistance gene, such as kanamycin resistance gene can be used. A loxP-KanMX-loxP cassette can advantageously be used for this purpose.

For the homologous recombination replacing the whole DNA sequence or part thereof of a S. cerevisiae gene, DNA fragments are used wherein the marker gene is flanked at the 5′- and 3′-ends by sequences which are homologous to the 5′- and 3′-ends of the studied S. cerevisiae gene.

Different processes can be used for the preparation of the corresponding DNA fragments which are also appropriate for the deletion of any specific S. cerevisiae gene. A linear DNA-fragment is used for the transformation of the suitable S. cerevisiae strain. This fragment is integrated into the S. cerevisiae genome by homologous recombination. These processes include:

1. “Conventional method” for the preparation of deletion cassettes (Rothstein, R. J. (1983) Methods in Enzymology, Vol. 101, 202-211).

2. “Conventional Method” using the PCR technique (“modified conventional method”).

3. SFH (short flanking homology)—PCR method (Wach, A. et al. (1994) Yeast 10: 1793-1808; Gültner, U. et al. (1996) Nucleic Acids Research 24:2519-2524).

1. In the “conventional method” for the preparation of deletion cassettes in the S. cerevisiae genome, the gene to be studied is either already present in an appropriate vector or is integrated in such a vector. With this method, any pBR-pUC- and pBluescript®-derivates can be used for example. A major part of the target gene sequence is eliminated from the vector, for instance using appropiate restriction sites, conserving however the 3′- and 5′-regions of the studied gene inside the vector. The selected marker gene is integrated between the remaining regions.

2. In the modified form of this “conventional method”, PCR is used. This method allows amplification of the 3′- and 5′-terminal regions of the coding sequence of the studied S. cerevisiae gene. This method amplifies selectively both terminal regions of the studied gene, therefore, two PCR-reactions must be carried out for each studied gene, amplifying once the 5′-end, and once the 3′-end of the gene. The length of the amplified terminal DNA-fragment depends on the existing restriction sites. The amplified terminal ends of the studied gene have generally a length of 50 to 5000 base pairs (bp), preferably a length between 500 and 1000 bp.

As template for the PCR-reactions, genomic DNA of S. cerevisiae or wild-type genes can be used. The primer-pairs (a sense and an antisense primer, respectively) are constructed so that they correspond to the 3′-end and the 5′-end sequence of the studied S. cerevisiae gene. Especially, the primer is selected such as to allow its integration by way of appropriate restriction sites.

As vectors, pBR-pUC- and pBluescript®-derivates can be used. In particular vectors already containing a gene encoding the selection marker, are appropriate. In particular, vectors can be used, which contain genes of the selection marker HIS3, LEU2, TRP1 or URA3.

The DNA segments of the studied S. cerevisiae gene, r obtained by PCR, are integrated in the vector at both sides of the selection marker, so that subsequently, as in the “conventional method”, the selection marker is flanked on both ends by DNA sequences which are homologous to the studied gene.

3. Homologous recombination in S. cerevisiae takes place in a very efficient and precise manner and the length of the DNA sequence homologous to the studied S. cerevisiae gene flanking the selection marker gene can in fact be considerably shorter than with the “modified conventional method”. The flanking ends homologous to the studied S. cerevisiae gene need to present a length of only about 20-60 bp, preferably 30-45 bp. The SFH-PCR method is particularly advantageous as the laborious cloning step can be obviated.

A PCR reaction is carried out on a DNA-template containing the gene for the selection marker to be used, wherein the primers are constructed so that the DNA sequence of the sense primer is homologous to the 5′-end of the selection marker sequence and so that the primer presents in addition at its 5′-end a region of preferably 40 nucleotides, which corresponds to the 5′-terminal sequence of the studied S. cerevisiae gene. The antisense primer is constructed in an analogous manner, i.e. it is complementary to the 3′-end of the gene sequence of the selection marker, wherein this primer contains at its 5′-end a region of also preferably 40 nucleotides, which corresponds to the complementary strand of the 34-terminal coding sequence of the studied gene.

For the amplification of S. cerevisiae genes to be studied by the SFH-PCR method, vectors containing the gene for the auxotrophic marker or selection marker can be used. Especially, plasmid pUG6 is used as the template. This plasmid contains a loxP-KanMX-loxP cassette (Gültner, U. et al. (1996) Nucleic Acids Research 24: 2519-2524). In other terms, the Kanamycin resistance gene is flanked at both ends by a loxP sequence (loxP-KanMX-loxP cassette). This cassette is advantageous in that the Kanamycin resistance gene can be eventually eliminated from the S. cerevisiae genome after integration of the loxP-KanMX-loxP cassette into the S. cerevisiae gene to be studied. Cre-recombinase of bacteriophage P1 can be used for this purpose. Cre-recombinase recognizes the loxP sequences and induces elimination of the DNA located between the two loxP sequences by a homologous recombination process. As a result only one loxP sequence remains and the so-called marker regeneration occurs, i.e. the S. cerevisiae strain may be transformed again using the loxP-KanMX-loxP cassette. This is particularly advantageous, when at least two functionally similar genes are to be deleted in order obtain a lethal phenotype.

With the PCR-method, the PCR reaction primers are at the 3′-end a preferably 20 nucleotide long sequence, which is homologous to the sequence situated left and/or right of the loxP-KanMX-loxP cassette, and at the 5′-end a preferably 40 nucleotide long sequence, which is homologous to the terminal ends of the gene to be studied.

Using the three methods, one obtains linear deletion cassettes containing the gene encoding the selection marker, which is flanked on both sides by homologous sequences of the gene to be studied. The deletion cassettes are used for the transformation of diploid S. cerevisiae strains. The diploid strain S. cerevisiae CEN.PK2 (Scientific Research & Development GmbH, Oberursel) can be used for example for this purpose. [CEN.PK2 Mata/MAT α ura3-52/ura3-52 leu2-3, 112/leu2-3, 112his3Δ1/his3Δ1 trp1-289/trp1-289 MAL2-8^{c}/MAL2-8^{c} SUC2/SUC2]

The strain CEN.PK2 is prepared and cultivated using known methods (Gietz, R. D. et al. (1992) Nucleic Acids Research 8: 1425; Güldener, U. et al. (1996) Nucleic Acids Research 24:2519-2524).

The cells of the S. cerevisiae strain used are transformed according to known processes with an appropriate DNA quantity of the linear deletion cassette (e.g. Sambrook et al. 1989). Thereafter, the medium in which the cells are cultivated is replaced by a new medium, a so-called selective medium, which does not contain the corresponding amino acid (e.g. histidine, leucine or tryptophan) or nucleic base (e.g. uracil) or, when using a deletion cassette containing the kanamycin resistance gene, by a medium containing geneticin (G418®) (e.g. a complete medium (YEPD) containing geneticin). Alternatively, the transformed cells may be plated on agar plates prepared using the corresponding media. Thereby, one selects the transformed cells, in which a homologous recombination occured, since only those cells can grow under these modified conditions.

However, in most cases, only one of the two copies of the gene in the double chromosome set of a diploid S. cerevisiae strain is replaced by the DNA of the deletion cassette during the transformation, resulting in a heterozygote-diploid S. cerevisiae mutant strain, wherein one copy of the gene studied is replaced by a selection marker, while the other copy of the gene is maintained in the genome. This presents the advantage that in case of a deletion of an essential gene, due to the existence of the second copy of the essential gene, the mutant S. cerevisiae strain is still viable.

The proper integration of the deletion cassette DNA at the predetermined chromosomal gene locus (gene locus of the gene to be studied) may be checked by Southern-Blot Analysis (Southern, E. M. (1975) J. Mol. Biol. 98:503-517) or by diagnostic PCR analysis using specific primers (Güldener, U. et al. (1996) Nucleic Acids Research 24:2519-2524)

The genetic separation of individual diploid cells may be monitored by tetrad analysis. To this end, reduction division (meiosis) is induced in the diploid cells, especially heterozygote mutant strains, using known methods such as nitrogen impoverishment on potassium acetate plates (Sherman, F. et al. (1986) Cold Spring Harbor Laboratory Press, Cold Spring Harbor, N.Y.; Guthrie, C. and Fink, G. R. (1991) Methods in Enzymology, Vol 194. Academic Press, San Diego, 3-21; Ausubel, F. M. et al. (1987) Current Protocol in Molecular Biology John Wiley and Sons, Inc., Chapter 13). Meiosis results only in asci with four ascospores (segregated), which can be indivualized after partial enzymatic digestion of the ascospore wall with zymolyase (Ausubel et al. (1987)) by way of micromanipulators (e.g. SINGER). For example when a tetrad analysis is carried out on a heterozygote-diploid mutant strain in which an essential gene present in the double chromosome set is replaced on one chromosome by homologous recombination, then only two segregated ascospores are viable, namely those which carry the essential gene. The two remaining segregated ascospores are not viable because they lack the essential gene.

In order to check if the genes studied by this method are really essential or if the homologous recombination leads to an alteration of an essential gene adjacent to the gene locus of the gene studied, the heterozygote diploid S. cerevisiae mutant strain is transformed with a centromere plasmid containing said studied gene.

A tetrad analysis is carried out on the transformants. When four instead of two viable segregates are obtained, then the studied gene contained in the centromere plasmid can complement the defect of the two non-viable haploid S. cerevisiae cells/mutant strains, which demonstrates that the studied S. cerevisiae gene is essential.

Preferably, plasmids present in low copy number, e.g. one or two copies per cell are used as centromere plasmids. For example plasmids pRS313, pRS314, pRS315 and 35′, pRS316 (Sijkorski, R. S. and Hieter, P. (1989) Genetics 122: 19-27) or similar plasmids can be used for this purpose. Preferably, the studied genes are integrated in said plasmids including their 3′- and 5′-end non-coding regions.

Individual S. cerevisiae genes may be studied using the above-described method, their sequences being totally or partially known. The complete genomic sequence of S. cerevisiae was made accessible to the public via the WWW (World Wide Web) on Apr. 24, 1996.

Different possibilities exist to have access to the S. cerevisiae genomic DNA sequence via the WWW.

MIPS (Munich information Centre of Protein Sequence) Address http://speedy.mips.biochem.mpq.de/mips/yeast/

SGD (Saccharomyces Genome Database, Stanford) Address http://genome-www.stanford.edu/Saccharomyces

YPD (Yeast Protein Database, Cold Spring Harbor) Address http://www.proteome.com/YPDhome.html

The complete S. cerevisiae DNA sequence is also accessible via FTP (file transfer protocol) in Europe (e.g. at the address: ftp.mips.embnet.org) in the U.S.A. (address: genome-ftp.stanford.edu) or in Japan (address: ftp.nig.ac.jp)

The complete S. cerevisiae DNA sequence was published in Nature, special issue No 387, 1997.

90 essential genomic S. cerevisiae genes have been identified by this way. These essential genes are listed in table 1. Table 1 contains the systematic gene name of the essential genes (corresponding to the denomination under which the corresponding DNA sequences are accessible in databanks), the deleted nucleotides and the corresponding amino acids of the essential genes (position 1 is taken as reference, this latter corresponding to the A of the probable initiation codon ATG of the ORF). The deleted nucleotides correspond to nucleotides deleted in the gene and the deleted amino acids correspond to the amino acids missing in the encoded protein. Furthermore aa corresponds to the total number of amino acids present in the encoded protein. The numbers of deleted nucleotides do not necessarily correspond to 3 times the numbers of deleted amino acids; this is explained by the fact that a gene is bigger than the encoded reading frame for amino acids. YMR134w for example encodes a protein of 237aa, the deletion starts at nucleotide 5 (counting starts from ATG) and continues until nucleotide 740, this also includes part of the terminator region which does not encode aa, so the deletion of the aa starts from aa 2 until the end of the protein which is aa 237. Furthermore, the information available concerning the functions of respective genes or of the encoded proteins and/or homologies/similarities to other genes or proteins are indicated. The primers used for the PCR reaction to prepare the DNA fragments appropriate for the deletion of the genes are listed in table 2, where S1 and S2 are the forward and reverse primers, respectively, and the bold letters corresponding to the nucleotides of the respective gene.

The data of table 1 emphasize that despite the fact that the S. cerevisiae gene DNA sequences are known, very little is known today about the function, the characteristic properties of these genes, the essential function of these genes, or the proteins encoded by the same.

According to one embodiment of the method, essential genes of S. cerevisiae are used to identify corresponding functionally similar genes in other mycetes.

By functionally similar genes in other mycete species, is meant genes which have a function similar or identical to that of the identified essential genes of S. cerevisiae. Functionally similar genes in other mycetes may, but need not be homologous in sequence to the corresponding essential S. cerevisiae genes. Functionally similar genes in other mycetes may exhibit only moderate sequence homology at the nucleotide level to the corresponding essential S. cerevisiae genes. By moderate sequence homology it is meant in the present invention genes having a sequence identity, at the nucleotide level, of at least 50%, more preferably of at least 60% and most preferably of at least 70%.

In addition, functionally similar genes in other mycetes may, but need not encode proteins homologous in sequence to the proteins encoded by the essential S. cerevisiae genes. Functionally similar proteins in other mycetes may exhibit moderate protein sequence homology to the proteins encoded by the essential S. cerevisiae genes.

By moderate protein sequence homology is meant in the present invention proteins having a sequence identity, at the amino-acid level, of a least 40%, preferably of at least 50%, more preferably of at least 60% and most preferably of at least 70%.

Genes homologous in sequence may be isolated according to known methods, for example via homologous screening (Sambrook, J. et al. (1989) Molecular Cloning Cold Spring Harbor Laboratory Press, N.Y.) or via the PCR technique using specific primers from genomic libraries and/or cDNA libraries of the corresponding mycetes.

According to one embodiment, genes homologous in sequences are isolated from cDNA libraries. In order to find out functionally similar genes in other mycetes, mRNA is isolated from mycete species to be studied according to known methods (Sambrock et al. 1989) and cDNA is synthesized according to known methods (Sambrock et al. 1989; or cDNA synthesis kits, e.g. from STRATAGENE).

The prepared cDNA is directionally integrated in a suitable expression vector.

For example, synthesis of the first cDNA strand may be carried out in the presence of primers having appropriate restriction sites in order to allow a subsequent cloning in the proper orientation with respect to the expression vector promoter. As restriction sites, any known restriction site may be used. As a primer, for instance the following primer, 50 nucleotides long may be used:

5′-GAGAGAGAGAGAGAGAGAGAACTAGTXXXXXXTTTTTTTTTTTTTTT
TTT-3′

The sequence (X)₆ represents an appropriate restriction site, for example for XhoI.

After two-strand synthesis, the cohesive ends of the double stranded cDNA are filled (blunt end) and the cDNA ends are then ligated using a suitable DNA adaptor sequence. The DNA adaptor sequence should contain a restriction site which should be different from the restriction site used in the primer for the synthesis of the first cDNA strand. The DNA adaptor may comprise For example complementary 9- or 13-mer oligonucleotides, whose ends represent the cohesive end of a restriction site. These ends may be for example a EcoRI-site:

5′ XXXXXGGCACGAG 3′
3′ XCCGTGCTC 5′

The single-stranded X in the adaptor sequence represent the cohesive end of a restriction site.

The cDNA provided with corresponding adaptor sequences is then cleaved using restriction endonuclease, whose recognition site was used in the primer for the synthesis of the first cDNA strand, for example XhoI. The cDNA thus obtained would have according to this example 3′-XhoI and 5′-EcoRI protruding ends and could be therefore directionally integrated into an expression vector cleaved with XhoI and EcoRI.

As expression vectors, among others, E. coli/S. cerevisiae shuttle vectors, i.e. vectors usable in E. coli as well as in S. cerevisiae are suitable. Such vectors may then be amplified for instance in E. coli. As expression vectors, those which are present in a high copy number as well as those present in a low copy number in S. cerevisiae cells can be used. For this purpose, for example vectors selected in the group consisting of pRS423-pRS426 (pRS423, pRS424, pRS425, pRS426) and/or pRS313-pRS316 (pRS313, pRS314, pRS315, pRS316) (Sikorki, R. S. and Hieter, P. (1989) Genetics 122: 19-27; Christianson T. W. et al. (1992) Gene 110: 119-122) are suitable.

Expression vectors should contain appropriate S. cerevisiae promoters and terminators. In case they do not have, these elements, the corresponding promoters and terminators are inserted in such a way that a subsequent incorporation of the generated cDNA remains possible. Particularly suitable are the promoters of S. cerevisiae genes MET25, PGK1, TPI1, TDH3, ADHI, URA3. One may use promoters of the wild-type gene in non modified form as well as promoters which were modified in such a way that certain activator sequences and/or repressor sequences were eliminated. As terminators, for example the terminators of the S. cerevisiae genes MET25, PGK1, TPI1, TDH3, ADHI, URA3 are suitable.

According to another embodiment, genes homologous in sequence are isolated from genomic libraries. Genomic DNA libraries from mycetes can be prepared according to procedures known (for example as described in Current Protocols in Molecular Biology, John Wiley and Sons, Inc). For example, genomic DNA from mycetes can be prepared using known methods for yeast cell lysis and isolation of genomic DNA (for example commercially available kits from Bio101, Inc). The genomic DNA can be partially digested using a restriction enzyme such as Sau3AI and the fragments are size-selected by agarose gel electrophoresis. DNA fragments having for example a size of 5-10 kb are then purified by classical methods (as for example, using Gene Clean kit from Bio101) and inserted in a E. coli/yeast shuttle vector such as YEP24 (described e.g. by Sanglard D., Kuchler K., Ischer F., Pagani J-L., Monod M. and Bille J., Antimicrobial Agents and Chemotherapy, (1995) Vol.39 No11, P2378-2386) cut by a restriction enzyme giving compatible ends (for example BamHI for Sau3AI-cut genomic DNA). The resulting expression library can be amplified in E. coli. However any known method, appropriate for the preparation of a genomic library, can be used in the present invention.

In order to find the genes in the studied mycete species, which are functionally similar to essential genes of S. cerevisiae, one S. cerevisiae essential gene is placed under control of a regulated promoter, either as an integrative (1) or extrachromosomal (2) gene.

1. For the integration of a regulated promoter in the S. cerevisiae genome, one replaces the native promoter of the selected essential gene by the regulated promoter, for example by homologous recombination via PCR (Güldener et al. (1996). The homologous recombination via PCR can be carried out for example in the diploid S. cerevisiae strain CEN.PK2. The successfull integration into one chromosome can be checked in haploid cells following tetrad analysis.

Using the tetrad analysis, one obtains four viable ascospores, wherein in two haploid segregates, the selected essential gene is placed under the control of the native promoter, while the essential gene in the two remaining segregates is placed under the control of the regulated promoter.

The last mentioned haploid segregates are used for the transformation with the cDNA or the genomic DNA present in the recombinant vector.

2. Using the extrachromosomal variant, the selected essential S. cerevisiae gene, is first inserted in a suitable expression vector, for example a E. coli/S. cerevisiae shuttle vector. For this purpose, the essential gene may be amplified via PCR from genomic S. cerevisiae DNA starting from the ATG initiation codon up to and including the termination codon. The primers used for this purpose may be constructed in such a way that they contain recognition sites for appropriate restriction enzymes, facilitating a subsequent insertion under control of a regulated promoter in an expression vector.

The recombinant expression vector with the plasmid copy of the essential S. cerevisiae gene under the control of a regulated promoter is subsequently used for the transcomplementation of the corresponding mutant allele. The corresponding mutant allele may be selected from the heterozygote-diploid mutant strains prepared by eliminating, partially or totally, by homologous recombination an essential mycete gene listed in table 1 (first column of table 1), as described above.

The expression vector with the selected essential S. cerevisiae gene is transformed in the corresponding heterozygote-diploid mutant strain carrying instead of the selected essential S. cerevisiae gene, a selection marker gene. The transformants are isolated by selection based on the auxotrophic marker contained in the expression vector used. The thus transformed heterozygote-diploid mutant strains are submitted to a tetrad analysis. One obtains four viable segregates. By retracing the corresponding markers of the mutant allele and the expression vector, the transformed wild-type segregates may be distinguished from segregates which do not contain the genomic copy of the essential gene. Segregates, which do not contain the genomic copy of the selected essential gene, are designated as trans-complemented haploid mutant strains. They are subsequently used for transformation with cDNA or genomic DNA libraries from other mycete species present in appropriate vectors.

As regulated promoters, inducible or repressible promoters may be used. These promoters can consist of naturally and/or artificially disposed promoter sequences.

As regulated promoters, for example the promoters of GAL1 gene and the corresponding promoter derivatives, such as for example promoters, whose different UAS (upstream activation sequence) elements have been eliminated (GALS, GALL; Mumberg, J. et al. (1994) Nucleic Acids Research 22:5767-5768) may be used. As regulated promoters, promoters of gluconeogenic genes may also be used, such as e.g. FBP1, PCK1, ICL1 or parts therefrom, such as e.g. their activation sequence (UAS1 and/or UAS2) or repression sequence (URS, upstream repression sequence) (Niederacher et al. (1992), Curr. Genet. 22: 636-670; Proft et al. (1995) Mol. Gen. Gent. 246: 367-373; Schüiler et al. (1992) EMBO J; 11: 107-114; Guarente et al. (1984) Cell 36: 503-511).

A S. cerevisiae mutant strain modified in this manner can be cultivated under growth conditions, in which the regulated promoter is active, so that the essential S. cerevisiae gene is expressed. The S. cerevisiae cells are then transformed with a representative quantity of the library containing the studied mycete species cDNA or genomic DNA. Transformants express additionally the protein whose coding sequence is present in the recombinant vector.

The method contemplates that the growth conditions may be modified in such a way as to inhibit the regulated promoter, under the control of which is the selected essential gene. Especially, growth conditions may be changed by replacing the growth medium. When for example the GAL1 promoter or a derivate thereof is used, one can replace the galactose-containing medium (induced state) by a glucose-containing medium (repressed state).

These modified conditions are lethal for the S. cerevisiae cells in which the recombinant vector does not carry the functionally similar genomic DNA or cDNA of the studied mycete species. On the contrary, the S. cerevisiae cells in which the recombinant vector expresses a functionally similar coding sequence of the studied mycete species, are viable, since in these cells the lethal metabolic defect is complemented by the protein encoded by the functionally similar gene.

The method contemplates that the recombinant vector (the plasmid) is isolated from the surviving transformants using known method (Strathern, J. N. and Higgins, D. R. (1991). Plasmids are recovered from yeast into Escherichia coli shuttle vectors in: Guthrie, C. and Fink, G. R. Methods in Enzymology, Volume 194. Guide to yeast genetic and molecular Biology. Academic Press, San Diego, 319-329) and the cDNA or genomic DNA is analyzed using DNA-analysis methods such as DNA sequencing. (Sanger et al. (1977), Proc. Natl. Acad. Sci. USA 74: 5463-5467)

The method contemplates that essential S. cerevisiae genes may be used for the identification of functionally similar genes and/or genes homologous in sequence in other mycetes, especially essential genes functionally similar and/or homologous in sequence in mycetes pathogenic to human, animal and plants. For this purpose for example mycetes of the classes phycomycetes or eumycetes may be used, in particular the subclasses basidiomycetes, ascomycetes, especially mehiascomycetales (yeast) and plectascales (mould fungus) and gymnascales (skin and hair fungus) or of the class of hyphomycetes, in particular the subclasses conidiosporales (skin fungus) and thallosporales (budding or gemmiparous fungus), among which particularly the species mucor, rhizopus, coccidioides, paracoccidioides (blastomyces brasiliensis), endomyces (blastomyces), aspergillus, penicilium (scopulariopsis), trichophyton (ctenomyces), epidermophton, microsporon, piedraia, hormodendron, phialophora, sporotrichon, cryptococcus, candida, geotrichum and trichosporon.

Of particular interest is the use of Candida Spp. especially Candida albicans, Candida glabrata, Aspergillus Spp., especially Aspergillus fumigatus, Coccidioides immitis, Cryptococcus neoformans, Histoplasma capsulatum, Blastomyces dermatitidis, Paracoccidioides brasiliens and Sporothrix schenckii.

The method contemplates that essential mycete genes are used to identify substances which may inhibit partially or totally the functional expression of these essential genes and/or the functional activity of the encoded proteins. Substances may be identified in this fashion, which inhibit mycetes growth and which can be used as antimycotics, for example in the preparation of drugs.

A particular feature of this method is that essential mycete genes or the corresponding encoded proteins are used as targets for the screening of the substances. The method contemplates that essential S. cerevisiae genes as well as functionally similar genes and/or genes homologous in sequence of other mycetes or the corresponding encoded proteins may be used as targets.

According to one embodiment of the screening method of the invention, myceces cells are provided, which contain the essential gene used as target, and those cells are incubated with the substance to be tested. By this way, the growth inhibitory effect of this substance with respect to the essential target gene is determined.

The mycetes cells which express the essential target gene to a different degree are used, and these cells are then incubated with the substance to be tested and the growth inhibitory effect of this substance is determined.

The method includes the use of two or more mycetes cells, or strains derived therefrom, which differ in that they express the essential target gene to a different degree.

For example, two, three, four, five, ten or more mycetes cells or the corresponding mycetes strains may be comparatively analysed with respect to the growth inhibitory effect of a substance used in a defined concentration. Through such concentration series, antimycotic substances may be distinguished from cytotoxic or inactive substances.

A particular embodiment of the method includes the use of haploid mycetes cells/strains for the screening, especially haploid S. cerevisiae cells/strains.

The method contemplates the integration of the essential gene selected as a target in a suitable expression vector.

As expression vectors E. coli/S. cerevisiae shuttle vectors are for example suitable. Especially vectors differing in their copy number per cell may be used. Therefore, one may use vectors, which are present in the transformed S. cerevisiae cells in a high copy number, or one can also use those with a low copy number. One embodiment comprises the use of expression vectors which allow the integration of the target gene in the S. cerevisiae genome.

For example the vectors pRS423, pRS424, pRS425, pRS426, pRS313, pRS314, pRS315, pRS316, pRS303, pRS304, pRS305, pRS306 (Sikorki and Hieter, 1989; Christianson et al. 1992) are appropriate as expression vectors.

The vectors of the series pRS423-pRS426 are present in a high copy number, about 50-100 copies/cell. On the contrary, the vectors of the series pRS313-pRS316 are present in a low copy number (1-2 copies/cell). When expression vectors from these two series are used, then the target gene is present as an extrachromosomal copy. Using the vector of the series pRS303-pRS306 allows the integration of the target genes into the genome. Using these three different expression vector types allows a gradual expression of the studied functionally similar essential gene.

The method includes that the growth inhibitory effect of substances with respect to mycetes cells/strains is comparatively determined using expression vectors differing for instance in the copy number of the vector/cell.

Such cells may express the essential target gene to a different degree and may exhibit a graduated reaction with respect to the substance.

The method includes also, that a target gene expression of different strength is obtained in different mycetes cells (regulated overexpression) by insertion of the target gene in the expression vector between specific selected S. cerevisiae promoters and terminators. S. cerevisiae promoters which are constitutively expressed, but with different strength, are suitable. Examples for such promoters are native promoters of S. cerevisiae genes MET25, PGK1, TPI1, TDH3, ADH1, URA3, TRP1, as well as corresponding derivatives therefrom, for example promoter derivatives without specific activator and/or repressor sequences.

Regulated promoters are also appropriate for the graduated over-expression of the target gene. The native promoters of the GALL genes and/or corresponding derivates thereof, for example promoters, in which different UAS elements have been eliminated. (GALS, GALL; Mumberg et al. (1994) Nucleic Acids Research 22: 5767-5768) as well as promoters of gluconeogenic genes, for example the promoters FBP1, PCK1, ICL1, or parts thereof, for example their activator-(UAS1 or UAS2) or repressor-(URS) sequences are used in corresponding non activable and/or non repressible test promoters (Schüller et al. (1992) EMBO J. 11: 107-114) Guarente et al. (1984) Cell 36: 503-511; Niederacher et al. (1992) Curr. Genet. 22: 363-370; Proft et al. (1995) Mol. Gen. Genet. 246: 367-373).

In the expression vector terminator for example the terminator sequence of S. cerevisiae genes MET25, PGK1, TPI1, TDH3, ADHI, URA3 may be used.

The method includes that by the use of cleverly selected expression vector types and/or the preparation of suitable expression vectors, eventually using promoters of different strength and differently regulated promoters, a series of expression vectors may be constructed, all containing the same target gene, but differing in that they express the target gene to a different extent.

The method includes the transformation of the expression vector in haploid wild-type cells of S. cerevisiae. The thus obtained S. cerevisiae cells/strains are cultivated in liquid medium and incubated in the presence of different concentrations of the tested substance and the effect of this substance on the growth behaviour of the cells/strains expressing the target gene to a different degree is comparatively analysed. The method also includes that haploid S. cerevisiae cells/strains, transformed using the respective expression vector type without target gene, are used as a reference.

The method includes that the screening of the substances can be carried out in different media using regulated promoters, especially GAL1 promoter and its derivates (GALS and GALL), since the expression degree may be largely influenced by the choice of the respective medium. Thus, the expression degree of the GAL1 promoter decreases in the following fashion: 2% galactose>1% galactose+1% glucose>2% glycerine>2% glucose.

The effect of the substances inhibiting the growth of wild-type cells of S. cerevisiae, may be partially or totally compensated by the overexpression of the functionally similar gene of another mycete species.

According to one embodiment, the method for screening antimycotic substances is carried out in vitro by contact of an essential or functionally similar gene or the corresponding encoded protein with the substance to be tested and determination of the effect of the substance on the target. Any in vitro test appropriate for determining the interaction of two molecules, such as a hybridization test or a functional test, can be used (e.g. enzymatic tests which are described in details in Bergmeyer H. U., Methods of Enzymatic Analysis, VCH Publishers). If the screening is carried out using the encoded protein as the target, then the corresponding essential gene is inserted by any suitable method known in the art, such as PCR amplification using a set of primers containing appropriate restriction sites, (Current Protocol in Molecular Biology, John Wiley and Sons, Inc) into an expression system, such as E. coli, Baculovirus, or yeast, and the expressed protein is then completely or partially purified by a method known in the art. Any purification method appropriate for the purification of expressed proteins, such as affinity chromatography can be used. If the target protein function is known, a functional test can then be carried out in which the effect of the antimycotic substance on the protein function is determined. If the protein function is unknown, substances which can interact with the target protein, e.g. which bind to the encoded protein, can be tested. In such a case a test such as protection of the target protein from enzymatic digestion by appropriate enzymes can be used.

The method also includes the identification of genes which are functionally similar and/or homologous in sequence to essential S. cerevisiae genes from humans, animals or plants. The corresponding human, animal or plant genes may optionally be used as target genes in the method in order to test if antimycotic substances exhibit an effect on these target genes.

A particular advantage of the method is that in this way substances may be identified which efficiently Inhibit mycetes growth and also the influence of these substances on corresponding functionally similar genes and/or genes homologous in sequence to essential S. cerevisiae genes from human, animal or plants may be determined.

The method includes also the possibility to check the existence of functionally similar genes and/or human, animal, or plant genes homologous in sequence to the corresponding essential mycete genes, for example by checking homology of the identified essential mycete genes or parts thereof with human, animal or plant sequence genes available in data banks. In this way, it is possible to select at an early stage from the identified essential mycete genes, depending on the aim, those for which no functionally similar gene and/or no human gene homologous in sequence exist, for example.

Thereby, the method offers a plurality of possibilities to identify selectively substances with antimycotic effects, with no harmful effect on human beings.

For example, it is possible to identify substances usable for the preparation of drugs for the treatment of mycosis or prophylaxis in immunodepression states. These substances may be used for example for the manufacture of drugs usable for the treatment of mycotic infections, which occur during diseases like Aids or Diabetes. Substances which may be used for the fabrication of fungicides, especially of fungicides which are harmless for humans and animals, can also be identified.

Furthermore, the method offers the possibility to identify antimycotic substances, which selectively inhibit growth of specific mycete species only.

The screening method is particularly advantageous inasmuch as it is sufficient to know whether the genes are essential, one does not need any additional information regarding the function of the essential genes or the function of the encoded proteins. In addition, it is particularly advantageous for the identification of functionally similar genes to essential S. cerevisiae gene, in other mycetes where the DNA sequence is not available for many of these genes.

### EXAMPLES

### Example 1

Preparation of a Deletion Cassette for ORF YML114c, by the Classical Method Using PCR (Modified Classical Method)

1) Construction of the Plasmids

pBluescript®KS+vector (Stratagene; the sequence of which is available on Genbank®X52327) is used as the starting vector for the preparation of the other plasmids.

The vector is cleaved with NotI and the single-stranded ends are subsequently eliminated by incubation with Mung Bean exonuclease. By religation of DNA fragments, the pKS+ΔNotI vector is thus obtained (corresponding to the pBluescript®KS+without the NotI restriction site).

pKS+ΔNotI is cleaved with PstI and BamHI and the DNA oligonucleotide, synthesized from the pK3/pK4 primer pair described below, is ligated in the opened plasmid. The pKS+neu plasmid thus prepared contains between PstI and BamHI restriction sites, the following novel restriction sites NotI, StuI, SfiI and NcoI (i.e. PstI-NotI-StuI-SfiI-NcoI-BamHI)

| | |
|---|---|
| 5′-GCGGCCGCAAGGCCTCCATGGCCG-3′ | PK3 |
| 5′-GATCCGGCCATGGAGGCCTTGCGGCCGCTGCA-3′ | PK4 |

The URA3 gene of S. cerevisiae is amplified via PCR, by use of the primer-pair PK9 and PK10, described below, and an Ycplac33 vector DNA (Gietz, R. D. and Sugino, A. (1988) 17 Gene 74: 527-534) as matrix. The amplified DNA is cleaved with BamHI and NotI and subsequently inserted in pKS+neu which has been cleaved by BamHI and NotI. The plasmid thus obtained is named pPK9/10.

| | |
|---|---|
| ..NotI.. | |
| 5′-ATCTGCAGCGGCCGCAAACATGAGAATTGGGTAATAACTG-3′ | PK9 |
| PstI | |
| ..SfiI.. | |
| 5′-ATGGATCCGGCCATGGAGGCCTTCAAGAATTAGCTTTTCAATTCATC-3′ | PK10 |
| BamHI | |

2) Preparation of the Deletion Cassette

The 5′-region of ORF YML114c was amplified by PCR using genomic DNA of S. cerevisiae as template and both primers YML114c-Asp718 and YLM114c-EcoRI, described below.

| | |
|---|---|
| YML114c-Asp718: | 5′-GCTGGTACCCGTCGGTCTCTTTACC-3′ |
| YLM114c-EcoRI: | 5′-TTGGAATTCATTGCCCTTTATGAGTCC-3′ |

The PCR fragment was subsequently cut with the restriction enzymes Asp718 and EcoRI. The resulting 613BP fragment was inserted in pPK9/10 linearized with Asp718 and EcoRI generating plasmid pYML114c-A.

The 3′region of ORF YML114c was amplified by PCR using genomic DNA of S. cerevisiae as template and both primers YML114c-BamHI and YLM114c-SacI, described below.

| | |
|---|---|
| YML114c-BamHI: | 5′-ATCGGATCCGCCAACAATGACAGCG-3′ |
| YLM114c-SacI: | 5′-GTTGAGCTCTGAGCGTTTGTCCTTG-3′ |

The PCR fragment was subsequently cut with BamHI and SacI. The resulting 535 bp fragment was inserted in plasmid pYML114c-A linearized with BamHI and SacI generating pYML114c-B.

This latter plasmid was used for transformation of S. cerevisiae after linearization with Asp178 and SacI.

### Examples 2-90

### Construction of Deletion Cassettes for the Remaining Genes Listed in Table 1

Using the method disclosed in example 1, the deletion cassettes of each of the essential genes can be constructed using as primers those disclosed in table 2.

### Example 91

S. cerevisiae cells from strain CEN.PK2 are transformed using each about 5 μg DNA of the linear deletion cassette of examples 1 to 90 according to known methods (Gietz et al. 1992; Güldener et al. 1996). The transformation reaction medium is plated on plates on the corresponding selective media. In this manner, the transformants are selected, in which homologous recombination occured, since only these cells can grow under these modified conditions.

The recombinant cells were submitted to a tetrad analysis in the following conditions: Reduction division (meiosis) was induced in the heterozygote mutant strain using known methods (Guthrie C. and Fink, G. R. (1991) Methods in Enzymology, Vol 194, Academic Press, San Diego). The resulting asci were submitted to partial enzymatic digestion with zygmolyase to digest the ascospore wall and separated using a micromanipulator (SINGER Instruments). This analysis demonstrated that all the above-mentioned 90 genes are essential for the growth of S. cerevisiae.

The present invention also applies more specifically to the following genes: YML114c, YLR186w, YLR215c, YLR222c, YLR243w, YLR272c, YLR275w, YLR276c, YLR317w, YLR359w, YLR373c, YLR424w, YLR437c, YLR440c, YML023c, YML049c, YML077w, YML093w, YML127w, YMR032w, YMR093w, YMR131c, YMR185w, YMR212c, YMR213w, YMR218c, YMR281w, YMR288w, YMR290c, YMR211w, YMR049c, YMR134w, YDR196c, YDR299w, YDR365c, YDR396w, YDR407c, YDR416w, YDR449c, YDR472w, YDR499w, YDR141c, YDR324c, YDR325w, YDR398w, YDR246w, YDR236c, YDR361c, YDR367w, YDR339c, YDR413c, YDR429c, YDR468c, YDR489w, YDR527w, YDR288w, YDR201w, YDR434w, YDR181c, YDR531w, YPL126w, YPL093w, YPL063w, YPL024w, YPL020c, YPL112w, YPL007c, YPL233w, YPL146c, YIL091c, YIL083c, YIL019w, YIL104c, YFL024c, YFR003c, YFR027w, YFR042w, YIR010w, YPR048w, YPR072w, YPR082c, YPR085c, YPR105c, YPR112c, YPR137w, YPR143w, YPR144c and YPR169w.

TABLE 1
ESSENTIAL GENES

| Systematic | | deleted | deleted | |
|---|---|---|---|---|
| ORF name | aa | nucleotides | amino acids | comments |
| YMR049c | 807 | 18-2277 | 6-759 | weak similarity to A. thaliana PRL1 protein |
| YMR134w | 237 | 5-740 | 2-237 | hypothetical protein |
| YDR196c | 241 | 174-543 | 59-181 | similarity to C. elegans hypothetical protein T05G5.5 |
| YDR299w | 534 | 41-1560 | 14-520 | hypothetical protein; nuclear localization (see |
| | | | | http://paella.med.yale.edu/YGAC/genes localization.html) |
| YDR365c | 628 | 45-1384 | 16-462 | weak similarity to Streptococcus M protein |
| YDR396w | 166 | 141-460 | 48-154 | hypothetical protein |
| YDR407c | 1289 | 48-3810 | 17-1270 | weak similarity to Myolp |
| YDR416w | 859 | 151-2540 | 51-847 | synthetic lethal with CDC40 |
| YDR449c | 440 | 21-1270 | 8-424 | hypothetical protein |
| YDR472w | 283 | 41-810 | 14-270 | similarity to P. falciparum 41-2 protein antigen |
| YDR499w | 747 | 41-2100 | 14-700 | weak similarity to hypothetical C. elegans protein, |
| | | | | M. genitalium peptide chain release factor 1 and YJL149w |
| YDR141c | 1698 | 51-4850 | 18-1617 | hypothetical protein |
| YDR324c | 717 | 79-2288 | 27-763 | weak similarity to beta transducin from S. pombe and |
| | | | | other WD-40 repeat containing proteins |
| YDR325w | 1051 | 110-3109 | 37-1037 | hypothetical protein |
| YDR398w | 643 | 41-1880 | 14-627 | similarity to human KIAA0007 gene |
| YDR246w | 219 | 41-580 | 14-194 | hypothetical protein |
| YDR236c | 218 | 30-489 | 11-163 | similarity to hypothetical A. thaliana protein |
| YDR361c | 283 | 43-812 | 15-271 | hypothetical protein |
| YDR367w | 221 | 354-643 | 119-215 | hypothetical protein |
| YDR339c | 189 | 40-529 | 14-177 | weak similarity to hypothetical protein YOR004w |
| YDR413c | 191 | 81-500 | 28-167 | weak similarity to NADH dehydrogenase; or YDR412w |
| YDR429c | 274 | 86-645 | 29-215 | TIF35; Vornlocher, H. -P., Hanachi, P. and Hershey, J. W. B. |
| | | | | Cloning and Characterization of the Two Large Subunits |
| | | | | of Yeast Translation Initiation Factor eIF3. |
| | | | | Unpublished; translation initiation factor eIF3 (p33 |
| | | | | subunit) |
| YDR468c | 224 | 123-602 | 42-201 | TLG1; member of the syntaxin family of t-SNAREs; tlg |
| | | | | mutants seems to have a defect in the retrieval pathway |
| | | | | to the TGN; viable |
| YDR489w | 294 | 131-630 | 44-210 | hypothetical protein |
| YDR527w | 439 | 41-1260 | 14-420 | weak similarity to Plasmodium yoelii rhoptry protein, or |
| | | | | YDR526c |
| YDR288w | 303 | 41-800 | 14-267 | hypothetical protein |
| YDR201w | 165 | 130-319 | 43-107 | hypothetical protein |
| YDR434w | 534 | 41-1400 | 13-467 | Similarity to S. pombe hypothetical protein |
| YDR181c | 481 | 194-1323 | 65-441 | SAS4 (viable); involved in silencing at telomeres |
| YDR531w | 367 | 41-850 | 14-284 | Similarity to hypothetical A. thaliana and C. elegans |
| | | | | proteins |
| YLR186w | 252 | 4-750 | 2-250 | strong similarity to S. pombe hypothetical protein |
| | | | | C18G6.07C |
| YLR215c | 360 | 31-970 | 11-324 | Similarity to rat cell cycle progression related D123 |
| | | | | protein; there are few domains identical to the D123 |
| | | | | protein |
| YLR222c | 817 | 8-2378 | 3-793 | similarity to Dip2p |
| YLR243w | 272 | 41-700 | 14-234 | strong similarity to YOR262w |
| YLR272c | 1176 | 15-3384 | 6-1128 | similarity to hypothetical human ORF |
| YLR275w | 110 | 32-360 | 11-90 | contains intron; strong similarity to human snRNPchain |
| | | | | D2 involved in systemic lupus erythematosus; identified |
| | | | | as part of the U1 complex by mass spectrometrie, PNAS |
| | | | | 94: 385-390 (1997) Neubauer G. et al. |
| YLR276c | 594 | 44-1733 | 15-578 | similarity to RNA helicases; identified as part of the |
| | | | | U1 complex by mass spectrometrie, PNAS 94: 385-390 |
| | | | | (1997) Neubauer G. et al. |
| YLR317w | 144 | 4-403 | 2-135 | — |
| YLR359w | 482 | 120-1399 | 41-467 | strong similarity to adenylosuccinate lyase |
| YLR373c | 901 | 14-2693 | 5-898 | similarity to hypothetical protein YGR071c |
| YLR424w | 708 | 109-2098 | 37-700 | weak similarity to Stulp |
| YLR437c | 133 | 7-376 | 3-126 | — |
| YLR440c | 709 | 18-1978 | 7-660 | — |
| YML023c | 556 | 81-1640 | 28-547 | weak similarity to Nmd2p |
| YML049c | 1361 | 258-3967 | 87-1323 | weak similarity to monkey UV-damaged DNA-binding protein |
| YML077w | 159 | 41-390 | 13-130 | — |
| YML093w | 899 | 29-2642 | 9-881 | similarity to P falciparum liver stage antigen LSA-1 |
| YML114c | 510 | 11-1410 | 3-470 | — |
| YML127w | 581 | 65-1704 | 21-568 | weak similarity to Los1p |
| YMR032w | 669 | 46-2002 | 15-668 | weak similarity to S. pombe cdc15 |
| YMR093w | 513 | 41-1300 | 13-434 | weak similarity to Pwp2p |
| YMR131c | 511 | 11-1410 | 3-470 | similarity to human retinoblastoma-binding protein |
| YMR185w | 981 | 65-2914 | 21-972 | — |
| YMR212c | 782 | 56-2287 | 18-763 | weak similarity to myosin |
| YMR213w | 590 | 58-1533 | 19-511 | similarity to S. pombe putative transcription factor |
| | | | | cdc5 |
| YMR218c | 1102 | 157-3253 | 52-1085 | — |
| YMR281w | 304 | 26-760 | 8-254 | — |
| YMR288w | 971 | 131-2670 | 43-890 | strong similarity to S. pombe und C. elegans proteins |
| YMR290c | 505 | 11-1471 | 3-491 | strong similarity to Myc-regulated DEAD box protein |
| YMR211w | 475 | 72-1341 | 25-447 | weak similarity to beta tubulins |
| YFL024c | 832 | 47-2406 | 16-802 | EPL1 (viable); weak similarity to YMR164c and Gal11p |
| YFR003c | 155 | 106-315 | 36-105 | hypothetical protein |
| YFR027w | 281 | 40-649 | 14-217 | hypothetical protein |
| YFR042w | 200 | 344-873 | 115-291 | hypothetical protein |
| YIL091c | 721 | 44-1953 | 15-651 | weak similarity to spt5p |
| YIL083c | 365 | 46-1005 | 16-335 | hypothetical protein |
| YIL019w | 346 | 81-1000 | 27-334 | hypothetical protein |
| YIL109c | 926 | 42-2721 | 14-907 | SEC24 (lethal); component of COPII coat of ER-Golgi |
| | | | | vesicles |
| YIL104c | 507 | 133-1082 | 45-361 | similarity to hypothetical S. pombe protein |
| YIR010w | 576 | 41-1500 | 14-500 | hypothetical protein |
| YIR015w | 144 | 85-274 | 29-92 | hypothetical protein |
| YPL126w | 896 | 41-2700 | 14-900 | weak similarity to fruit fly TFIID subunit p85 |
| YPL093w | 647 | 151-1900 | 51-634 | similarity to M. jannaschii GTP-binding protein, |
| | | | | GTP1/OBG-family, weak similarity to other GTP-binding |
| | | | | proteins |
| YPL063w | 476 | 126-1385 | 42-462 | similarity to hypothetical protein YLR019w, YLL010c and |
| | | | | S. pombe hypothetical protein SPAC2F7.02c |
| YPL024w | 241 | 41-550 | 14-184 | NCE4 (viable); negative regulator of CTS1 expression |
| YPL020c | 621 | 44-1813 | 15-605 | weak similarity to Smt4p |
| YPL012w | 1228 | 41-3630 | 14-1210 | hypothetical protein |
| YPL007c | 588 | 55-1614 | 19-538 | hypothetical protein |
| YPL233w | 216 | 41-610 | 14-204 | hypothetical protein |
| YPL146c | 455 | 46-1325 | 16-442 | weak similarity to myosin heavy chain proteins |
| YPR048w | 623 | 41-1650 | 14-550 | similarity to M. domestica NADPH-ferrihemoprotein |
| | | | | reductase and mammalian nitric-oxide synthases |
| YPR072w | 560 | 42-1541 | 14-514 | NOT5 (viable); component of the NOT protein complex |
| YPR082c | 143 | 140-279 | 47-93 | weak similarity to Ypk1p |
| YPR085c | 448 | 277-1166 | 93-389 | hypothetical protein |
| YPR105c | 861 | 74-2543 | 25-848 | hypothetical protein |
| YPR112c | 887 | 52-2521 | 18-841 | similarity to RNA-binding proteins |
| YPR137w | 573 | 41-1680 | 14-560 | weak similarity to Taf90p |
| YPR143w | 250 | 41-710 | 14-237 | hypothetical protein |
| YPR144c | 552 | 107-1616 | 36-539 | similarity to YDR060w and C. elegans hypothetical protein |
| YPR169w | 514 | 201-1490 | 201-1490 | hypothetical protein |

TABLE 2
Primers used for gene deletions

| | |
|---|---|
| Name | Sequence 5′-3′ |

Gene deletions on chromosome 13

| | |
|---|---|
| YDR472w-S1 | ATG TCT CAA AGA ATA ATT CAA CCA AGC GCA TCT GAC |
| | CAA CCA GCT GAA GCT TCG TAC GC |
| YDR472w-S2 | AGC CAA ATC TCA AAC CTT CCC TGT CAA GCA CTT GCC |
| | TGT CGC ATA GGC CAC TAG TGG ATG TG |
| YDR499w-S1 | ATG AGA CGA GAA ACG GTG GGT GAA TTT TCT TCA GAT |
| | GAC GCA GCT GAA GCT TCG TAC CC |
| YDR499w-S2 | CGT ACT TTA CTT GCA TTA TTC TCC CCG TTC TTT TAT |
| | TCA AGC ATA GGC CAC TAG TGG ATG TG |
| YMR049c-S1 | CAG ACT ATT GAT TAC TTT ATG ACC GGT TAG TTT CTT |
| | TAG TCA GCT GAA GCT TCG TAC GC |
| YMR049c-S2 | TCT GTT CTA ACA TAA CTA GGT CAA TGA TGG CTA AGA |
| | ACA AGC ATA GGC CAC TAG TGG ATC TG |
| YMR134w-S1 | GCA AAG TGT GGT ATA GAA AAA GAA CCA AAG GCC GGT |
| | ATG TCA GCT GAA GCT TCG TAC GC |
| YMR134w-S2 | TGT GTG TGT GCC TAC CTG CAT GTA TGC ATT TAG CAA |
| | TTG AGC ATA GGC CAC TAG TGG ATC TG |
| YML023c-S1 | CAC GCA ATG GTG CAC ATT ATT TTG TTG AAC TCA CTG |
| | AGA ACA GCT GAA GCT TCG TAC GC |
| YML023c-S2 | ATT AGT TAC TTA TTC TAT AAT TAC ACT TTT ATC ATG |
| | AAC GGC ATA GGC CAC TAG TGG ATC TG |
| YML049c-S1 | AAT TCC TGC TCA TTC AAG GAA AGT CTC AGG AAA TTT |
| | TCA CCA GCT GAA GCT TCG TAC GC |
| YML049c-S2 | ACT CCT GCA TCG GAC ACT TCG TCG ATC TGG AAG CAG |
| | GGT CGC ATA GGC CAC TAG TGG ATC TG |
| YML077w-S1 | ATG GGG ATA TAT TCA TTT TGG ATC TTT GAT AGG CAT |
| | TGT ACA GCT GAA GCT TCG TAC GC |
| YML077w-S2 | TTC TAT TGG TGA TCT TTC TTG TCC CTT GAC CTC TCA |
| | TTT CGC ATA GGC CAC TAG TGG ATC TG |
| YML093w-S1 | GCT AAC TTA AAT ATG GCA AAA AAG AAA TCT AAG AGC |
| | AGA TCA GCT GAA GCT TCG TAC GC |
| YML093w-S2 | CAA AGG ATC AAT AAC TTG GCC TGG CTT AGT CAT GAT |
| | TCT CGC ATA GGC CAC TAG TGG ATC TG |
| YML114c-S1 | AAC GTG TAA TTG AGG GAC TCA TAA AGG GCA ATG ACT |
| | TCC ACA GCT GAA GCT TCG TAC GC |
| YML114c-S2 | GAC TTG TAG TAG CAT CGA TAT TGG TTG TGT TAT GTG |
| | CTA CGC ATA GGC CAC TAG TGG ATC TG |
| YML127w-S1 | CCG CTA AAT GGT ACT CCA GTA AGC GAG GCA CCC GCC |
| | ACA ACA GCT GAA GCT TCG TAC GC |
| YML127w-S2 | ATA ACC CCG ACG TGT TTT CCA TGT ATT CAG ACA ATG |
| | CTA AGC ATA GGC CAC TAG TGG ATC TG |
| YMR032w-S1 | CTA CAG TTA TGA AGC TTC TTT TTG GGA CCC AAA CGA |
| | CAA TCA GCT GAA GCT TCG TAC GC |
| YMR032w-S2 | CAG AAA ACT AGT AAA ATT GAT ATA CAT CGA GAT CAA |
| | AGA CGC ATA GGC CAC TAG TGG ATC TG |
| YMR093w-S1 | ATG TCG ACT GCT AGG CCT AGA ATA ATC ACT TCG AAG |
| | GCT CCA GCT GAA GCT TCG TAC GC |
| YMR093w-S2 | AAG CAC CAA TTC AGT AGC GGC TCT AAT GTA GAT TCA |
| | TCT CGC ATA GGC CAC TAG TGG ATC TG |
| YMR131c-S1 | CTT TAA CTT CCT TTT GCC AGT GAA CAA ACA ATA ATT |
| | GTG GCA GCT GAA GCT TCG TAC GC |
| YMR131c-S2 | GGT CTA TCG AGG TCA ACG AGG AAC AAG ATA GAG TGG |
| | TCT CGC ATA GGC CAC TAG TGG ATC TG |
| YMR185w-S1 | ATC AAC ATA CAC GAT ATA TTG AAT ACA AGA CCG AAG |
| | CTC ACA GCT GAA GCT TCG TAC GC |
| YMR185w-S2 | GTA ATG GGT TAT AAA CTA TCT AGT ACG GTT AAA AGC |
| | TTG TGC ATA GGC CAC TAG TGG ATC TG |
| YMR212c-S1 | CCT CTT GAA CTT AAA GAA TGT AAA TCT TCA TTT GCG |
| | TCT TCA GCT GAA GCT TCG TAC GC |
| YMR212c-S2 | CGG ATG ATG TTC ACA CCA AAA CAT CAG AAA CTG GTC |
| | AAT CGC ATA GGC CAC TAG TGG ATC TG |
| YMR213w-S1 | ATA CGT GAA AGG CGG TGT ATG GAC CAA TGT GGA GGA |
| | TCA GCA GCT GAA GCT TCG TAC GC |
| YMR213w-S2 | GCT GTA ACT GTT CAA TAG ACT CCA CTT TTG ATT GGA |
| | TCG AGC ATA GGC CAC TAG TGG ATC TG |
| YMR218c-S1 | GAC TCA AAT GCA TTA GAG TGA TCA ACT CTA CAA CTT |
| | TTA CCA GCT GAA GCT TCG TAC GC |
| YMR218c-S2 | GAA GGC ATT TGA CGG AAC TGT ACG AAC GCT TAA CAG |
| | GCT TGC ATA GGC CAC TAG TGG ATC TG |
| YMR281w-S1 | CTG AAG AAA AGT TAA ATG AAG ATG TTG AGG CGT ACA |
| | AAG GCA GCT GAA GCT TCG TAC GC |
| YMR281w-S2 | AGT ACG TAT TGT GCA TGT GTA TTC ATA AGT GAA AGC |
| | TTG TGC ATA GGC CAC TAG TGG ATC TG |
| YMR288w-S1 | GAA AAC CTG CAG AAA GAA GCT GCA CGT ATT GGT GAG |
| | AAC GCA GCT GAA GCT TCG TAC GC |
| YMR288w-S2 | CCA AAC CTT CTA AAA TAC GCA TAA TAG CAT GTG GTG |
| | AAG TGC ATA GGC CAC TAG TGG ATC TG |
| YMR290c-S1 | TGA GTT TTA CGT CTT TTG GTA TTT GGC GTT TTT CCA |
| | CTG GCA GCT GAA GCT TCG TAC GC |
| YMR290c-S2 | GAT AAG CTG AGC AAT ATT AAC AGG AGA ACT ATG GCT |
| | ACC CGC ATA GGC CAC TAG TGG ATC TG |
| YMR211w-S1 | AGA GAG CAA ACC ATT TGA CTA CTC AAT TCT TCA ATA |
| | TAC ACA GCT CAA GCT TCG TAC GC |
| YMR211w-S2 | ATT TCA ATC ATC TTA CTC CGT GAA TCA GGT TCG GAA |
| | TGA TGC ATA GGC CAC TAG TGG ATC TG |

Gene deletions on chromosome 4

| | |
|---|---|
| YDR196c-S1 | ATG CTT ATG ATC AAA TTG TGT TAT ACT TCA AGG ACA |
| | AAA TCA GCT GAA GCT TCG TAG GC |
| YDR196c-S2 | TTT CAA TCT GTT CGT ATA AGT CAA CCA ATG TGC TGT |
| | TAT TGC ATA GGC CAC TAG TGG ATC TG |
| YDR299w-s1 | ATG GAA AAA TCA CTA GCG GAT CAA ATT TCC GAT ATC |
| | GCC ACA GCT GAA GCT TCG TAC GC |
| YDR299w-S2 | CAA AGA TTT GGA TAT CAT CGT TTT TAA CAG CCT CTA |
| | ATT CGC ATA GGC CAC TAG TGG ATC TG |
| YDR365c-S1 | CTG GAG AGA ACC CAA AGA AGG AAG GTG TAG ATG CTA |
| | GGT TCA GCT GAA GCT TCG TAC GG |
| YDR365c-S2 | TTA GTA TGC TTT TTA TTA ACA GAT TTC AAC TTG CTT |
| | TTC TGC ATA GGC CAC TAG TGG ATC TG |
| YDR396w-S1 | CAG ATA CAC TAT TGT GGT GTA ATC TGG ACC TTG ACT |
| | GTC TCA GCT GAA GCT TCG TAC GC |
| YDR396w-S2 | TAG AGA AAA CAC TGA ATG ATC TTA GCG ACC GTA CAA |
| | AAG AGC ATA GGC CAC TAG TGG ATC TG |
| YDR407c-S1 | TTC TTA AGC ATT TCC CAA GCT ATG TTG GCC CAT CTA |
| | AGA TCA GCT GAA GCT TCG TAC GC |
| YDR407c-S2 | AAT AAC AGA CAA GAT AAC GTT TTC AGA GTC GAA CTG |
| | GAT TGC ATA GGC CAC TAG TGG ATC TG |
| YDR416w-S1 | ACT TAC ATG GAA AAG ATA TAT CGA GTA TTG GAA AGA |
| | GGA GCA GCT GAA GCT TCG TAC GC |
| YDR416w-S2 | TCA AAT ATC TAG TTC TAT TTC ATC TGG ATT AAT CGA |
| | ATA TGC ATA GGC CAC TAG TGG ATC TG |
| YDR449c-S1 | CAC ATC ACC GAT TTC TAA TAA TGT CGA AGA CAA GAT |
| | ACT ACA GCT GAA GCT TCG TAC GC |
| YDR449c-S2 | ATA ATT AAA TCT AGA ATT TTA TAC CTA GGA TCA TCT |
| | TCT GGC ATA GGC CAC TAG TGG ATC TG |
| YDR141c-S1 | TTC GTA ATC TTT GAA TTC TGC GAT TTC ATC TAC CAG |
| | CGC GCA GCT GAA GCT TCG TAC GC |
| YDR141c-S2 | CAC TAA AGC CCC TTA CAA TTG ACT CAA ATA ATA AAC |
| | AAC TGC ATA GGC CAC TAG TGG ATC TG |
| YDR324c-S1 | AAG AAG CCT GAA AAT ACG AAA CAA ACC GGT GAA GAT |
| | GAC CCA GCT GAA GCT TCG TAC GC |
| YDR324c-S2 | AAA CACTAA CTT TGG TTG AAT AAA CGC CTT TTG TTT |
| | GGA GGC ATA GGC CAC TAG TGG ATC TG |
| YDR325w-S1 | GAC ATT AAT ACG AAA ATC TTT AAC TCA GTT GCT GAA |
| | GTA TCA GCT GAA GCT TCG TAC GC |
| YDR325w-S2 | ACC TCG CTG AAA GAC TCT GAA TCC TTA TCT TCT TCA |
| | TCT AGC ATA GGC CAC TAG TGG ATC TG |
| YDR398w-S1 | ATG GAT TCT CCT GTT CTA CAG TCC GCT TAT GAC CCA |
| | TCA GCA GCT GAA GCT TCG TAC GC |
| YDR398w-S2 | AAC GTC ACT ATA TCC GGC TTC CTC CTC GCC GTC GCT |
| | CTG CGC ATA GGC CAC TAG TGG ATC TG |
| YDR246w-S1 | ATG GCC ATC GAA ACA ATA CTT GTA ATA AAC AAA TCA |
| | GGC GCA GCT GAA GCT TCG TAC GC |
| YDR246w-S2 | AAC AGG TTA GAT CTT ATA GGC ATT TCC ATT GAG TAA |
| | GAT GGC ATA GGC CAC TAG TGG ATC TG |
| YDR236c-S1 | CTA AAA TAT TGA ACT TGA CCC TGG CCC CAT AAA AAT |
| | CAT TCA GCT GAA GCT TCG TAC GC |
| YDR236c-S2 | TTG AAG TGT TGA TGT TTACGT GGA CTA TTT ATG TTT |
| | CGT TGC ATA GGC CAC TAG TGG ATC TG |
| YDR361c-S2 | TTA CCA AGT GGA AAT TTC TGT TTC CAA TTC ATC GAT |
| | ACT TGC ATA GGC CAC TAG TGG ATC TG |
| YDR361c-S1 | GGT TCA AGC TAT CAA ATT AAA TGA TTT APA AAA TAG |
| | GAA GCA GCT GAA GCT TCG TAC GC |
| YDR367w-S1 | ATC TGC GTA CTT TAT ACA ATC GAT ACC ATT TCC ACT |
| | TGT TCA GCT GAA GCT TCC TAC GC |
| YDR367w-S2 | GTT TTG TTC TAC GTC ATC CCT ATC AAC TAA ATA TTT |
| | GGG GGC ATA GGC CAC TAG TGG ATC TG |
| YDR339c-S1 | TAT GGG TAA AGC TAA GAA AAC AAG AAA GTT TGG CCT |
| | CGT ACA GCT GAA GCT TCG TAC GC |
| YDR339c-S2 | TAA AAG ACA TCT GGC AAT TTT TCA ATG ACG TAT GCG |
| | TGA CGC ATA GGC CAC TAG TGG ATC TG |
| YDR413c-S1 | TTC TTT GGT TTA TTC TTC GTT CAT TTT TGG TCA AAT |
| | ATC TCA GCT GAA GCT TCG TAC GC |
| YDR413c-S2 | ACA AAA GAA AGC ACA AGA GTT TAT TAA GGA GCA GGA |
| | AAG GGC ATA GGC CAC TAG TGG ATC TG |
| YDR429c-S1 | TCT AGA TCT ATC ATT ACA TAC AAG ATT GAA GAC GGT |
| | GTC ACA GCT GAA GCT TCG TAC GC |
| YDR429c-S2 | TTT CTT TGT TTC TAA CGA CAG AAA CTC TTG GAA TGG |
| | GTG CGC ATA GGC CAC TAG TGG ATC TG |
| YDR468c-S1 | GTC ACA ATA CTG CTG GTG ATG ACG ATC AAG AGG AGG |
| | AAA TCA GCT GAA GCT TCG TAC GC |
| YDR468c-S2 | CAA GAC GAC AAT AAG AAG TCC TAT ACA ACA ATC GTC |
| | GTA TGC ATA GGC CAC TAG TGG ATC TG |
| YDR489W-S1 | ACT ACC CAC AGA GAT GCA AAT ACA ATA GTG GGT TCG |
| | TCC TCA GCT GAA GCT TCG TAC GC |
| YDR489w-S2 | AGT CGG GCT CAT CTA TCA TGT TTA CGC TAC CTT CTG |
| | TAT CGC ATA GGC CAC TAG TGG ATC TG |
| YDR527w-S1 | ATG GAC TTA CTG GGC GAT ATA GTG GAG AAA GAT ACA |
| | TCT GCA GCT GAA GCT TCG TAC GC |
| YDR527w-S2 | CCC CAC CGC CTT GTT TCC ATA ACC AAA GTG CAT CAA |
| | TAG CGC ATA GGC CAC TAG TGG ATC TG |
| YDR288w-S1 | ATG AGT TCT ATA GAT AAT GAC AGC GAT GTG GAT TTA |
| | ACA GCA GCT GAA GCT TCG TAC GC |
| YDR288w-S2 | GCC CAT GAT TTC TTG CAC CAA TTT TTC AAG AGA CTC |
| | TAG TGC ATA GGC CAC TAG TGG ATC TG |
| YDR201w-S1 | CCC ATG TCT GGA CTA TTC AGA GCA TCA TCG TCA TCC |
| | ATA CCA GCT GAA GCT TCG TAC GC |
| YDR201w-S2 | AAA AGG GTT TTC CGT TTA GTT CCC GAA TAT GAT GTT |
| | GAA AGC ATA GGC CAC TAG TGG ATC TG |
| YDR434w-S1 | ATG TCC AAT GCA AAT CTA AGA AAA TGG GTT GGT TTT |
| | TGC TCA GCT GAA GCT TCG TAC GC |
| YDR434w-S2 | TAA AGG TAA ATA CAC AGC TAT CAT GTG CTC TTG TGG |
| | GAA GGC ATA GGC CAC TAG TGG ATC TG |
| YDR181c-S1 | AGG ATA AAC CCA AAT GCT GGA CAT CTA AGG AAA TCT |
| | AAG TCA GCT GAA GCT TCG TAC GC |
| YDR181c-S2 | TAG TTG GGT TTG AAT CGT TAT CAC GGG AGA ACA TTG |
| | CTT TGC ATA GGC CAC TAG TGG ATC TG |
| YDR531w-S1 | ATG CGG CGA ATT ACT CAA GAG ATA TCT TAC AAT TGC |
| | GAT TCA GCT GAA GCT TCG TAC GC |
| YDR531w-S2 | AAA TAA GCT ATT TGC CCA ATA TTG TTG GAG ATG GCG |
| | AAT AGC ATA GGC CAC TAG TGG ATC TG |

Gene deletions on chromosome 12

| | |
|---|---|
| YLR186w-S1 | CTA GTC ACC AAG AAG AAA ACC CGT AAA ATC GTA GGT |
| | CAT GCA GCT GAA GCT TCG TAC GC |
| YLR186w-S2 | ATA CAA AGA GGA TGC CAA GTA GAC TTA AAC ACT ATA |
| | AAA TGC ATA GGC CAC TAG TGG ATC TG |
| YLR215c-S1 | TTA CTT ATT GAT GTC CTC ACA AGA ATA TAC AAC TTT |
| | TAT ACA GCT GAA GCT TCG TAC GC |
| YLR215c-S2 | AGC TCT CGG ATT GCT TCA GGA TTT AAA CTA GCT TCT |
| | ACG AGC ATA GGC CAC TAG TGG ATC TG |
| YLR222c-S1 | CTC TCA ACG GTA GTA AGC CAT ACT ACG TAC AAT ATG |
| | GAT CCA GCT GAA GCT TCG TAC GC |
| YLR222c-S2 | AAT ATG TAA CTT TGT TCA ACT AAG TTA TCA ACC CTT |
| | GTG AGC ATA GGC CAC TAG TGG ATC TG |
| YLR243w-S1 | ATG TCT CGC GTT GGT GTC ATG GTA TTA GGA CCT GCA |
| | GGT GCA GCT GAA GCT TCG TAC GC |
| YLR243w-S2 | GAT AAT ATG GTT TCT ATA CTG TCA GGA TTA TTA GAT |
| | TCC AGC ATA GGC CAC TAG TGG ATC TG |
| YLR272c-S1 | TTT GGG TCT CGC ACT TTC TCA GTC TTC CAA CTA ATT |
| | TCT CCA GCT GAA GCT TCG TAC GC |
| YLR272c-S2 | GGT AAC TGA CTT CGT TAC TTT ATG AGA TGT CCG GCT |
| | TTA GGC ATA GGC CAC TAG TGG ATC TG |
| YLR275w-S1 | CCG TTT TAT CAT GTC GTA TGT TTG ATC TTA ACC ATT |
| | TTT ACA GCT GAA GCT TCG TAC GC |
| YLR275w-S2 | CAA CGA TAA CTG AAT CAC CTC TTA AGA ATA GTT TAC |
| | TTA TGC ATA GGC CAC TAG TGG ATC TG |
| YLR276c-S1 | CTT CAA CGG GTC TAC TTT ACC ATT CTT TGG CTT ACT |
| | GAC TCA GCT GAA GCT TCG TAC GC |
| YLR276c-S2 | AGC TAT GAG AAA AAG TCT GTG GAA GGC GCT TAT ATT |
| | GAC GGC ATA GGC CAC TAG TGG ATC TG |
| YLR317w-S1 | CTG CCA TCT TCT GCC ACC ACT TTG TCC TTC TTT CTT |
| | GAT GCA GCT GAA GCT TCG TAC GC |
| YLR317w-S2 | GAA GTA AAC TAA CTA GTA AAG TAG GCT AAT TCG AAA |
| | CGA TGC ATA GGC CAC TAG TGG ATC TG |
| YLR359w-S1 | GGC TAT TGC TGA GAA GGA ATT GGG CTT AAC TGT TGT |
| | TAC ACA GCT GAA GCT TCG TAC GC |
| YLR359w-S2 | AAC TTG ACT TGT TCA TCG TTT AGG TAC TTT TGG AAA |
| | GGT TGC ATA GGC CAC TAG TGG ATC TG |
| YLR373c-S1 | ACA CAC AGG TAC AGA GTG CTG AAA GAG GAT TGG TGT |
| | TGC CCA GCT GAA GCT TCG TAC GC |
| YLR373c-S2 | CAA ACA GAG TTT GTT CCT TTG TAT GTC CTA TGG AAG |
| | ATA CGC ATA GGC CAC TAG TGG ATC TG |
| YLR424w-S1 | GAC ATG ACA TAC ACT AAT GAT GCC TTG AAA ACT AGT |
| | AGC GCA GCT GAA GCT TCG TAC GC |
| YLR424w-S2 | ATA GGT ACT TTC TAG AGG TCA AGG GCC CAT AAA TAA |
| | ATT GGC ATA GGC CAC TAG TGG ATC TG |
| YLR437c-S1 | ATT GTG CAA GTC TGT TAA AGT CTT CTC TTG GAT CCA |
| | TTA ACA GCT GAA GCT TCG TAC GC |
| YLR437c-S2 | CAT CAC ACA CTA ATA CAG GAA CAA ACA AGA CTT AAT |
| | GGA CGC ATA GGC CAC TAG TGG ATC TG |
| YLR440c-S1 | TTG CCA AGA AAA TTG CAG TAA AAA TGT TGG AAG AGC |
| | AAC TCA GCT GAA GCT TCG TAC GC |
| YLR440c-S2 | GCT CCA ATT CTA GTG TGC TCC ATT GCG ATG TAA CAA |
| | TTT CGC ATA GGC CAC TAG TGG ATC TG |

Gene deletions on chromosome 6

| | |
|---|---|
| YFL024c-S1 | TGA TGA ATT TTT CTG GGT TAT AGA AGA GTT CTG TTT |
| | CCC TCA GCT GAA GCT TCG TAC GC |
| YFL024c-S2 | ACA CCT TCA AAC GCT ATA GAG ATC AAT GAC GGT TCG |
| | CAT AGC ATA GGC CAC TAG TGG ATC TG |
| YFR003c-S1 | TGT GGA AGA GGT TCC CGC AGT TTT GCA GCT TCG AGC |
| | AAC TCA GCT GAA GCT TCG TAC GC |
| YFR003c-S2 | ATC TTC TTT GTC TAC GTT CGT TAA AGT CAA GAT CCT |
| | TCT CGC ATA GGC CAC TAG TGG ATC TG |
| YFR027w-S1 | AAT GAA AGC TAG GAA ATC GCA GAG AAA AGC GGG CAG |
| | TAA ACA GCT GAA GCT TCG TAC GC |
| YFR027w-S2 | AAT TTG GTT GCG ATA CCC AAC TTC CTT GCT GTC CTG |
| | CAC AGC ATA GGC CAC TAG TGG ATC TG |
| YFR042w-S1 | AGT TTG CAC CAA TGG CAA TAT GCC TGT GAT AAA GAT |
| | AAG GCA GCT GAA GCT TCG TAC GC |
| YFR042w-S2 | CAT GGA AGT TAT TTG GTT GCT TAG ATT CCA CGG GTT |
| | CAA AGC ATA GGC CAC TAG TGG ATC TG |

Gene deletions on chromosome 9

| | |
|---|---|
| YIL109c-S1 | TGT CTC ATC ACA AGA AAC GTG TTT ACC CAC AAG CTC |
| | AGC TCA GCT GAA GCT TCG TAC GC |
| YIL109c-S2 | TCA TGA TTT GTA AGA ATT CTC TGT AAC TTT CGT TAT |
| | TCA AGC ATA GGC CAC TAG TGG ATC TG |
| YIL091c-S1 | AGT GAC AGT TCT GTG AGG GAA AAG AAT GAT AAT TTC |
| | CGT GCA GCT GAA GCT TCG TAC GC |
| YIL091c-S2 | CAT TGT AAA ATT CAG GAT TGT TTG GAG GCT TAT AAA |
| | AAA CGC ATA GGC CAC TAG TGG ATC TG |
| YIL083c-S1 | ACC TCT ACC CGT GCT CAA CAG ACC TCA AAT TCA TAC |
| | GTC TCA GCT GAA GCT TCC TAC GC |
| YIL083c-S2 | CGA TGA CTT CTG GGA TTA TCA TCT CTT CAA TGA TAT |
| | GGT GGC ATA GGC CAC TAG TGG ATC TG |
| YIL019w-S1 | TTC AAA GAA AAG CTT TTG AAA GTC AGT TCG GAT CTT |
| | TAG ACA GCT GAA GCT TCG TAC CC |
| YIL019w-S2 | TAG CGA CGG GAT TTC TTT TTG CCA TTA AAT TTA CCA |
| | CTC CGC ATA GGC CAC TAG TGG ATC TG |
| YIL104c-S1 | TCC CCT TAC TAT TTA AGA TTA AGA TTT CCT CAC GAA |
| | TTA ACA GCT GAA GCT TCG TAC GC |
| YIL104c-S2 | CCT GAT ACC TGT AAT GAT TGC AAT CTT GAT GAG AGA |
| | GCT GGC ATA GGC CAC TAG TGG ATC TG |
| YIR010w-S1 | ATG AGT CTG GAA CCC ACA CAA ACG GTC TCC GGT ACG |
| | CCG CCA GCT GAA GCT TCG TAC GC |
| YIR010w-S2 | ACC TTA GCT CAT CGA CTT TTG TTT CTA ACT TTG TCT |
| | CAA CGC ATA GGC CAC TAG TGG ATC TG |
| YIR015w-S1 | GCG AAC CAG GAC CAT TTC CAT AGA TTA AAC TAC CTC |
| | TAC CCA GCT GAA GCT TCG TAC GC |
| YIR015w-S2 | ATT TCC AGT TTT TTT GGG GTC CAT AAC AAC CGA TGG |
| | CAT TGC ATA GGC CAC TAG TGG ATC TG |

Gene deletions on chromosome 16

| | |
|---|---|
| YPL233w-S1 | ATG TCA CAA GGT CAG TCC AAA AAA CTG GAC GTA ACT |
| | GTT GCA GCT GAA GCT TCG TAC GC |
| YPL233w-S2 | CAA TCC TCC TCC AGG AAG TCC ATT AAG CGC TTG ACC |
| | TTT TGC ATA GGC CAC TAG TGG ATC TG |
| YPL146c-S1 | TCC AAC TAA TCT AACCAA GAA ACC ATC TGA ATA CAA |
| | ACA GCA GCT GAA GCT TCG TAC GC |
| YPL146c-S2 | TTT GAA GTC CTT ATG TGT CCA CTT TTC AGT GAT TTT |
| | CTG CGC ATA GGC CAC TAG TGG ATC TG |
| YPL126w-S1 | ATG ACG CAA TCC CTA GGT ATG GAA CAG TAT AAA CTG |
| | TCA GCA GCT GAA GCT TCG TAC GC |
| YPL126w-S2 | TAT GTT AAT ACT TTC ATC ACA CGA TCA AAA AAT GTA |
| | TCC AGC ATA GGC CAC TAG TGG ATC TG |
| YPL093w-S1 | CAA GAT TAC AAG AAT CAG AGCGTT CTA TAT GCG TAA |
| | AGT TCA GCT GAA GCT TCG TAC GC |
| YPL093w-S2 | CGG AAA TCT GTC TTA CGG ACA CCA CGC TTA CCA CTG |
| | AAT AGC ATA GGC CAC TAG TGG ATC TG |
| YPL063w-S1 | TTC AAG CAT CCA GAA GAC TTT TAA CAA GTT ATT CAA |
| | GTT TCA GCT GAA GCT TCG TAC GC |
| YPL063w-S2 | GGA TTC AGC AAT CTT CTT CTT TTT CTT TTC CTC TTC |
| | AAA TGC ATA GGC CAC TAG TGG ATC TG |
| YPL024w-S1 | ATG TCT TTT TCA TCT ATC TTA TCA CAG GAT ATC ACA |
| | GAT GCA GCT GAA GCT TCG TAC GC |
| YPL024w-S2 | ACT TGT GAG TCC TTC AAT ATG AAA ACG CCC CTA TTG |
| | AAC AGC ATA GGC CAC TAG TGG ATC TG |
| YPL020c-S1 | TCA GTT GAA GTA GAT AAG CAC CGG AAC ACA CTA CAG |
| | TAT CCA GCT GAA GCT TCG TAC GC |
| YPL020c-S2 | TCG GTT AAA ATC AAA TGG GCA ATA AAT CTT CTC ATC |
| | CTA AGC ATA GGC CAC TAG TGG ATC TG |
| YPL012w-S1 | ATG GAT CAA GAC AAA GTT GCT TTT CTT TTA GAG CTG |
| | GAG GCA GCT GAA GCT TCG TAC GC |
| YPL012W-S2 | ATT TGA ACT TTGGAC CTT TCT TAT TAT GTT TGC CAA |
| | TCT TGC ATA GGC CAC TAG TGG ATC TG |
| YPL007c-S1 | CCC GTT GTT TGC TCA CGG GAC ATA TCT TTA CAT GCG |
| | TTG TCA GCT GAA GCT TCG TAC GC |
| YPL007c-S2 | GGA CTT ATT GGT AGA TAG AAA GGA ATT TGA GGA TTG |
| | GAA GGC ATA GGC CAC TAG TGG ATC TG |
| YPR048w-S1 | ATG TCA TCG AGC AAG AAA ATC GTC ATC CTC TAT GGA |
| | TCG GCA GCT GAA GCT TCG TAC GC |
| YPR048w-S2 | AAT TAG TTA TTT CCT CAC CTA ATC TCC ATA AGT AGT |
| | CTT GGC ATA GGC CAC TAG TGG ATC TG |
| YPR072w-S1 | TGT CTC AAA GAA AGC TAC AAC AGG ATA TCG ATA AGC |
| | TTT TCA GCT GAA GCT TCG TAC GC |
| YPR072w-S2 | AGA TTT GCC CAT TCC TGG TGG TAA CTT TTC GAT TTC |
| | TTT AGC ATA GGC CAC TAG TGG ATC TG |
| YPR082c-S1 | CTT CGA TTG CTG AAA GAG TAA GGA ACT TTG CAG TTA |
| | TTT ACA GCT GAA GCT TCG TAC GC |
| YPR082c-S2 | CAA TAA AGT TCA ACT TGT TGT TGT TCC CTG TAC CAA |
| | AAT CGC ATA GGC CAC TAG TGG ATC TG |
| YPR085c-S1 | CTG TAC ATT CTT TCG AAA GAC TCC ATG CTG CGA ATT |
| | TTT GCA GCT GAA GCT TCG TAC GC |
| YPR085c-S2 | TCC CAC TTT ATA GTT ATG GGA TTT CGA GCT GGA TTC |
| | GGT AGC ATA GGG CAC TAG TGG ATC TG |
| YPR105c-S1 | AGC TCG ATC ATC GAG GGC GAA TTG TCT AAA AAT CTA |
| | GCA ACA GCT GAA GCT TCG TAC GC |
| YPR105c-S2 | CTG TGT TCT ATC AAT CTT CAT ATT TGT AGC TTT AAT |
| | TCT TGC ATA GGC CAC TAG TGG ATC TG |
| YPR112c-S1 | CAT TGT CAA GGG TTT GCC CGT CTA TCT AAC AGA TGA |
| | TAA TCA GCT GAA GCT TCG TAC GC |
| YPR112c-S2 | GAA ACC TTC GTT TTC TTC ATC ATC CAC ATC CAG TTT |
| | CTT TGC ATA GGC CAC TAG TGG ATC TG |
| YPR137w-S1 | ATG TCA GAT GTT ACC CAA CAG AAA AAG AGG AAA AGA |
| | TCC ACA GCT GAA GCT TCG TAC GC |
| YPR137w-S2 | AAA AGC CTG TTT GGT CAA TGA CAG CTG AAT ATA TAC |
| | CAT TGC ATA GGC CAC TAG TGG ATC TG |
| YPR143w-S1 | ATG GGC TCC AAG CAC AGA GTA GAC ACT AAG GAT AAG |
| | AAA ACA GCT GAA GCT TCG TAC GC |
| YPR143w-S2 | TTC ATT GTC GCT TCC TGC GGC AGC TTT AAC TAA ATC |
| | CAA AGC ATA GGC CAC TAG TGG ATC TG |
| YPR144c-S1 | TTC CAG AAA ATG TTA CTC AAT TGG AAG AAG ATG AGA |
| | CAG ACA GCT GAA GCT TCG TAC GC |
| YPR144c-S2 | CCA TGC TAC CCC AGG CAA GTA GAC GTT ACC TTG GGA |
| | TGA CGC ATA GGC CAC TAG TGG ATC TG |
| YPR169w-S1 | TTT TAG ATG CTG AAC TGC CCA TTA TAA TAA CTG GCT |
| | TTG GCA GCT GAA GCT TCG TAC GC |
| YPR169w-S2 | CTT CTT GAT CCC ATG CTC ATA CAG GTC GTT TTT TTT |
| | GTT GGC ATA GGC CAC TAG TGG ATC TG |

1-12. (cancelled)

13. A method for the screening of antimycotic substances wherein an essential gene from mycetes or a functionally similar mycete gene, or the corresponding encoded protein, is used as target and wherein the essential gene is selected from the group consisting in YML114c, YLR186w, YLR215c, YLR222c, YLR243w, YLR272c, YLR275w, YLR276c, YLR317w, YLR359w, YLR373c, YLR424w, YLR437c, YLR440c, YML023c, YML049c, YML077w, YML093w, YML127w, YMR032w, YMR093w, YMR131c, YMR185w, YMR212c, YMR213w, YMR218c, YMR281w, YMR288w, YMR290c, YMR211w, YMR049c, YMR134w, YDR196c, YDR299w, YDR365c, YDR396w, YDR407c, YDR416w, YDR449c, YDR472w, YDR499w, YDR141c, YDR324c, YDR325w, YDR398w, YDR246w, YDR236c, YDR361c, YDR367w, YDR339c, YDR413c, YDR429c, YDR468c, YDR489w, YDR527w, YDR288w, YDR201w, YDR434w, YDR181c, YDR531w, YPL126w, YPL093w, YPL063w, YPL024w, YPL020c, YPL012w, YPL007c, YPL233w, YPL146c, YIL091c, YIL083c, YIL019w, YIL109c, YIL104c, YFL024c, YFR003c, YFR027w, YFR042w, YIR010w, YIR015w, YPR048w, YPR072w, YPR082c, YPR085c, YPR105c, YPR112c, YPR137w, YPR143w, YPR144c and YPR169w.

14. The method of claim 13 wherein mycete cells which express the essential gene, or a functionally similar mycete gene, to a different level are incubated with the substance to be tested and the growth inhibiting effect of the substance is determined.

15. The method of claim 13 wherein said target gene or the corresponding target encoded protein is contacted in vitro with the substance to be tested and the effect of the substance on the target is determined.

16. The method according to claim 13 wherein the screened substances partially or totally inhibit the functional expression of the essential genes or the functional activity of the encoded proteins.

17. The method according to claim 14 wherein the screened substances partially or totally inhibit the functional expression of the essential genes or the functional activity of the encoded proteins.

18. The method according to claim 15 wherein the screened substances partially or totally inhibit the functional expression of the essential genes or the functional activity of the encoded proteins.

19. The method according to claim 13 wherein the mycete species are selected from the group comprising Basidiomycetes, Ascomycetes and Hyphomycetes.

20. The method according to claim 13, wherein said functionally similar genes are essential genes from Candida Spp, or Aspergillus Spp.

21. The method according to claim 20, wherein said functionally similar genes are essential genes from Candida albicans, or Aspergillus fumigatus.

22. The method according to claim 13 wherein the functionally similar genes are identified by:
a) providing a S. cerevisiae mutant strain in which the gene of S. cerevisiae to be investigated is either integrative or extrachromosomal under the control of a regulated promoter,
b) culturing said mutant strain under growth conditions in which the regulated promoter is active,
c) transforming the mutant strain with cDNA or genomic DNA that has been prepared from the mycete-species to investigate and that has been integrated into an appropriate vector,
d) altering the culture condition, so that the regulated promoter is switched off and only S. cerevisiae cells which contain a functionally similar gene can survive,
e) isolating and analyzing the cDNA or genomic DNA.

23. The method according to claim 22 wherein the functionally similar gene has a sequence identity, at the nucleotide level, with the corresponding S. cerevisiae essential gene of at least 50%, preferably of at least 60%, and most preferably of at least 70%.

24. The method according to claim 22 wherein the functionally similar gene encodes a protein having a sequence identity, at the amino-acid level, with the corresponding S. cerevisiae essential gene encoded protein of at least 40%, preferably of at least 50%, more preferably of at least 60% and most preferably of at least 70%.

25. The method according to claim 13 wherein said mycete cells are haploid S. cerevisiae cells.

26. The method according to claim 22 wherein said mycete cells are haploid S. cerevisiae cells.

27. The method according to claim 13 wherein the essential genes of S. cerevisiae are identified by integration through homologous recombination of a selection marker at the locus of the gene to be studied.

28. The method according to claim 25 wherein the essential genes of S. cerevisiae are identified by integration through homologous recombination of a selection marker at the locus of the gene to be studied.
